(19) United States
(12) Patent Application Publication
(10) Pub. No.: US 2025/0289929 A1
(43) Pub. Date: Sep. 18, 2025

(54) THERMALLY STABLE MONOMERS AND METHODS OF USE THEREOF

(71) Applicant: ARIZONA BOARD OF REGENTS ON BEHALF OF ARIZONA STATE UNIVERSITY, Scottsdale, AZ (US)

(72) Inventors: Jose Sintas, Phoenix, AZ (US); Timothy Long, Phoenix, AZ (US)

(21) Appl. No.: 19/082,032

(22) Filed: Mar. 17, 2025

Related U.S. Application Data
(60) Provisional application No. 63/566,009, filed on Mar. 15, 2024.

(51) Int. Cl. C08G 73/06; C07D 487/04; C09J 5/00; C09J 179/08; H01L 21/683

(52) U.S. Cl. CPC C08G 73/0672; C07D 487/04; C09J 5/00; C09J 179/08; H01L 21/6836; C09J 2203/326; C09J 2301/416

(57) ABSTRACT

The present invention relates to thermally stable monomers and methods of use thereof.

This application claims priority to U.S. Provisional Application No. 63/566,009 filed on Mar. 15, 2024, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The present invention relates to thermally stable monomers and methods of use thereof.

### BACKGROUND OF THE INVENTION

As competitive innovation continues in the semiconductor industry, the development of new materials capable of supporting technically challenging patterning processes continue to progress. These materials must meet diverse thermomechanical and optical requirements for multi-step fabrication processes, with an emphasis on thermal expansion coefficients, thermal stability, off-gassing, and photo-activity.

### SUMMARY OF THE INVENTION

Aspects of the invention are drawn towards a compound of Formula (I) or composition comprising the structure of Formula (I)

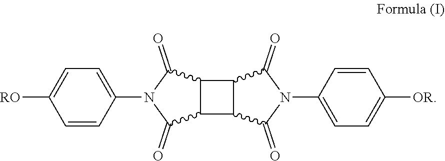

In embodiments, R is selected from the group consisting of H, alkenyl, or hydroxyalkyl. In embodiments, the hydroxyalkyl is selected from the group consisting of C₁-C₁₂ hydroxyalkyl, and the alkenyl is selected from the group of C₁-C₁₂-alkenyl. In embodiments, the composition is selected form the group consisting of:

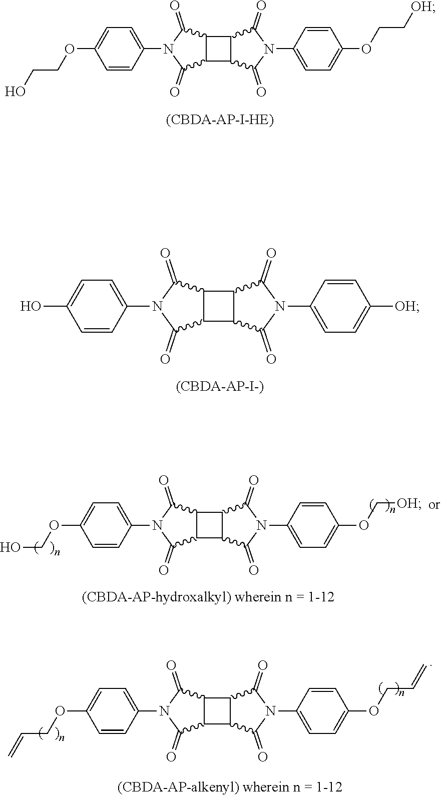

Aspects of the disclosure are drawn towards a polymer comprising the structure of Formula (I)

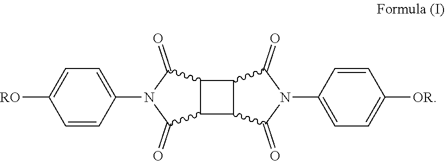

In embodiments, R is selected from the group consisting of H, alkenyl, or hydroxyalkyl. In embodiments, the polymer comprises a reneating unit of

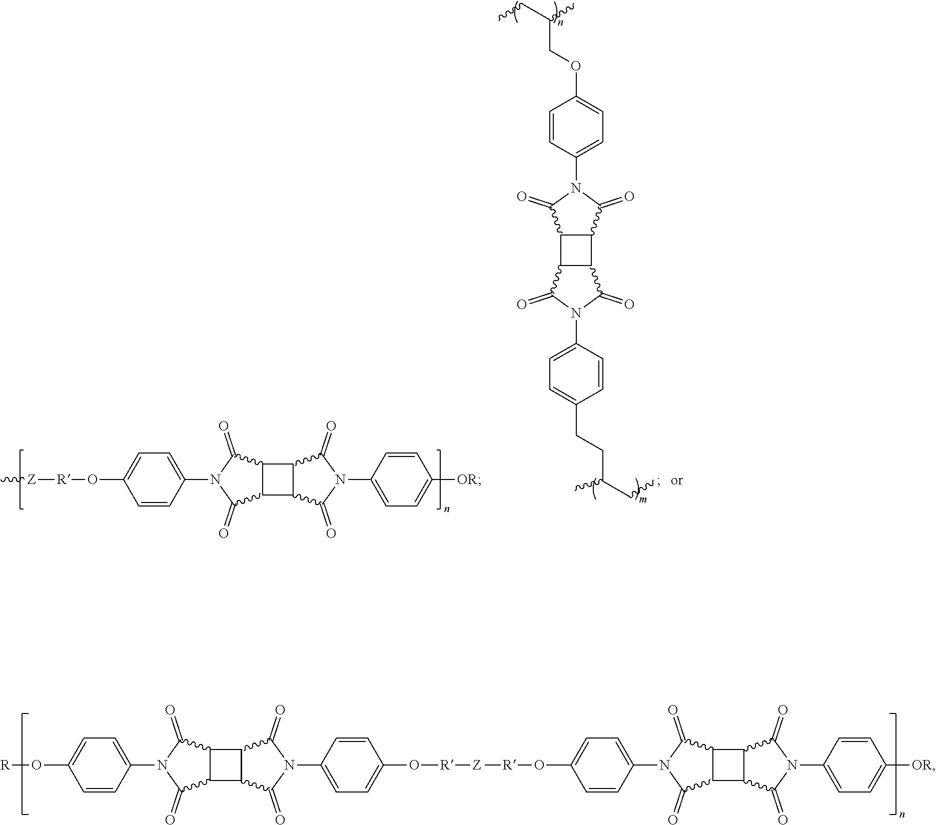

wherein, R is selected from the group consisting of H or hydroxyalkyl; R′ is absent when R is H, or R′ is-O-alkyl when R is hydroxyalkyl; and Z is a polyurethane, polyester, polysulfone, polyketone, or a monomer thereof.

In embodiments, the polymer comprises about 1 mol. % to about 99 mol. % of the structure of Formula (I). In embodiments, the polymer further comprises at least one co-monomer. In embodiments, the co-monomer is selected from the group consisting of: an isocyanate terminated monomer or polymer; a di-carboxylic acid; and

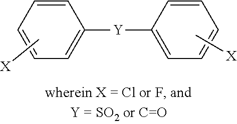

In embodiments, the co-monomer is

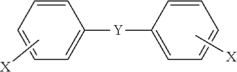

wherein X═Cl or F, and Y=SO₂ or C═O; and the polymer further comprises a diphenol co-monomer. In embodiments, the isocyanate terminated monomer or polymer is selected from the group consisting of:

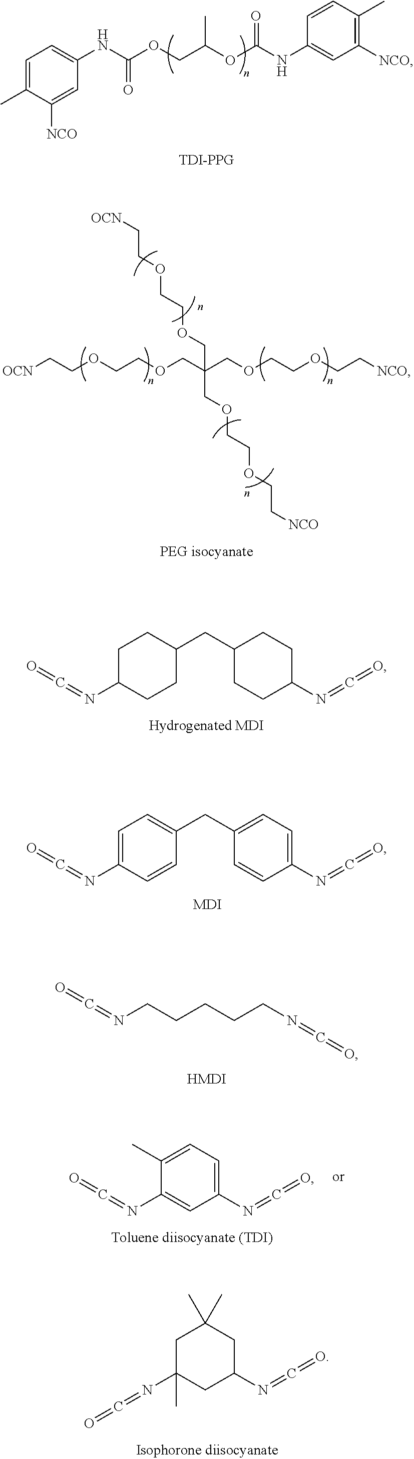

In embodiments, the di-carboxylic acid is selected from the group consisting of

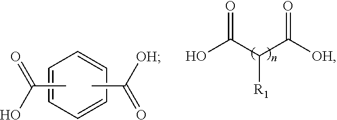

wherein R¹ is selected from the group consisting of H, alkyl, or aryl;

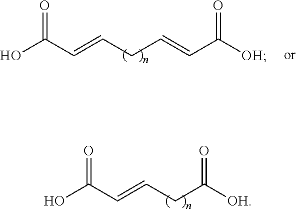

In embodiments, the co-monomer is

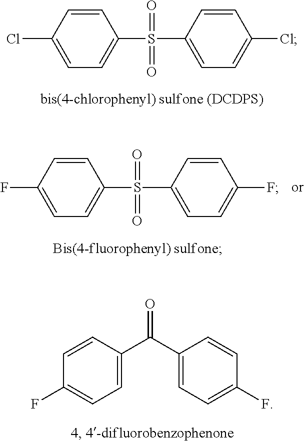 [SMILES: O=S(=O)(c1ccc(Cl)cc1)c1ccc(Cl)cc1 ; O=S(=O)(c1ccc(F)cc1)c1ccc(F)cc1 ; O=C(c1ccc(F)cc1)c1ccc(F)cc1]

In embodiments the diphenol is:

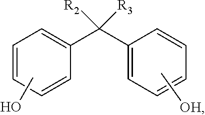

wherein R₂ and R₅ are independently selected from the group consisting of H and alkyl. In embodiments, the diphenol is

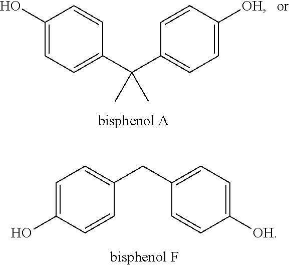 [SMILES: CC(C)(c1ccc(O)cc1)c1ccc(O)cc1 ; "or" ; Oc1ccc(Cc2ccc(O)cc2)cc1]

In embodiments, the polymer is a copolymer of the formula:

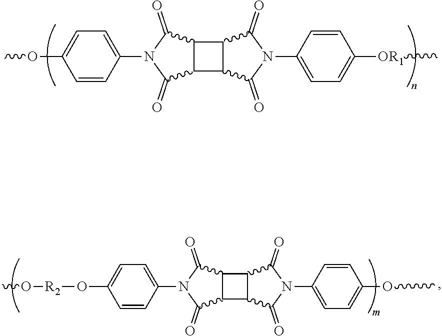

wherein R₁ and R₂ is any combination of a polyurethane, polysulfone, polyketone, polyester, or a monomer thereof.

Aspects of the disclosure are drawn towards a method of temporarily bonding a support substrate to a semiconductor wafer for processing, the method comprising: obtaining a device wafer and a support substrate; applying the photocleavable adhesive polymer of as described herein to the surface of the support substrate, thereby forming an adhesive layer; adhering the device wafer to the support substrate by placing it on top of the adhesive layer; processing the device wafer; and irradiating the adhesive layer with 254 nm light, thereby debonding the support substrate from the device wafer. In embodiments, the support substate comprises quartz or glass. In embodiments, the polymer is applied via spincoating.

Other objects and advantages of this invention will become readily apparent from the ensuing description.

### BRIEF DESCRIPTION OF THE FIGURES

FIG. 1 shows non-limiting, exemplary data. Rheological studies simulate a spin-coating application of CBDA-AP-I-HE and PPG monomers that display temporal stability at 25° C., however polymerize when exposed to elevated temperatures.

FIG. 2 shows non-limiting, exemplary data. DMA indicates phase separation of the CBDA-AP-I PU homopolymer.

FIG. 3 shows non-limiting, exemplary stress-strain curves. Stress-strain curves before and after irradiation demonstrates the effect of cyclobutane ring cleavage on tensile properties.

FIG. 4 shows non-limiting, exemplary ¹H NMR spectroscopy. ¹H NMR spectroscopy reveals appearance of maleimide protons in CBDA-AP-I-HE PU after 254 nm irradiation.

FIG. 5 shows non-limiting, exemplary ¹H NMR spectrum of CBDA-AP-I.

FIG. 6 shows non-limiting, exemplary ¹H NMR spectrum of CBDA-AP-I-HE.

FIG. 7 shows non-limiting, exemplary ¹H NMR spectrum of CBDA-AP.

FIG. 8 shows non-limiting, exemplary FTIR spectroscopy indicating complete imidization of CBDA-AP after thermal treatment to 300° C.

FIG. 9 shows step-wise TGA indicating two discrete weight-loss steps for thermal imidization of CBDA-AP.

FIG. 10 shows non-limiting, exemplary ¹H NMR end group analysis. Analysis displayed a disappearance of BPA end groups across all PSU compositions that contained CBDA-AP-I. This indicates PSU 2 and 3 contain CBDA-AP-I end groups.

FIG. 11 shows non-limiting, exemplary ATR FTIR spectroscopy. ATR FTIR spectroscopy probed incorporation of CBDA-AP-I into the PSU backbone through absorbances in the newly introduced imide carbonyl stretch.

FIG. 12 shows representative TGA of all PSUs inidicating an earlier onset weight loss for CBDA-AP-I containing PSUs.

FIG. 13 shows representative DSC indicating the effect of increased CBDA-AP-I loading on T_{g}.

FIG. 14 shows representative DMA indicating thermal transitions of synthesized PSUs. Incorporation of CBDA-AP-I results in decreased thermal performance that correlates with mol. % concentration.

FIG. 15 shows representative ¹H NMR displays purity of CBDA-AP-I.

FIG. 16 shows a photograph of the reaction product between CBDA-AP-I-HE and 20K 4-arm PEG isocyanate with 15 wt. % solids in DMSO, 0.7 wt. % CBDA-AP-I-HE, 14 wt. % PEG, and DABCO catalyst.

### DETAILED DESCRIPTION OF THE INVENTION

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower). In embodiments, the term “about” can be denoted by “˜”.

As used herein, the term “substantially the same” or “substantially” can refer to variability typical for a particular method is taken into account.

The terms “sufficient” and “effective”, as used interchangeably herein, can refer to an amount (e.g., mass, volume, dosage, concentration, and/or time period) needed to achieve one or more desired result(s).

Before explaining at least one embodiment of the disclosure in detail, it is to be understood that the disclosure is not necessarily limited in its application to the details set forth in the following description or exemplified by the examples. The disclosure can be used for other embodiments or of being practiced or carried out in various ways. Other compositions, compounds, methods, features, and advantages of the disclosure will be or become apparent to one having ordinary skill in the art upon examination of the following drawings, detailed description, and examples. All such additional compositions, compounds, methods, features, and advantages can be included within this description, and be within the scope of the disclosure.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g., have 5, 6 or 7 carbons in the ring structure. The term “alkyl” (or “lower alkyl”) as used throughout the specification, examples, and claims can include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein can refer to an alkyl group, as defined herein, but having from one to ten carbons, or from one to six carbon atoms in its backbone structure. Likewise, “lower alkenyl” and “lower alkynyl” have similar chain lengths. In some embodiments, alkyl groups are lower alkyls. In some embodiments, a substituent described herein as alkyl can be a lower alkyl.

It will be understood by those skilled in the art that the moieties substituted on the hydrocarbon chain can themselves be substituted, if appropriate. For instance, the substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined herein for alkyl groups.

The term “alkylthio” refers to an alkyl group, as defined herein, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined herein for alkyl groups.

The terms “alkenyl” and “alkynyl”, refer to unsaturated aliphatic groups analogous in length and possible substitution to the alkyls described herein, but that contain at least one double or triple bond respectively. As used herein, the term “alkenyl” can refer to an unsaturated branched, straight-chain, or cyclic alkyl radical having at least one carbon-carbon double bond derived by the removal of one hydrogen atom from a single carbon atom of a parent alkene. The group can be in either the cis or trans conformation about the double bond(s). In embodiments described herein, the alkenyl group can be C₂-C₁₃ alkenyl. Non-limiting examples of alkenyl groups include ethenyl, propenyl, butenyl, pentenyl, hexenyl, octenyl, etc.

The terms “alkoxyl” or “alkoxy” as used herein refers to an alkyl group, as defined herein, having an oxygen radical attached thereto. Representative alkoxyl groups include methoxy, ethoxy, propyloxy, and tert-butoxy. An “ether,” for example, can be two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by-O-aryl or O-heteroaryl, wherein aryl and heteroaryl are as defined herein. The alkoxy and aroxy groups can be substituted as described herein for alkyl.

The terms “amine” and “amino” are art-recognized and refer to both unsubstituted and substituted amines, e.g., a moiety that can be represented by the general formula:

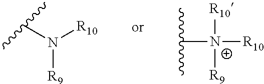

wherein R₉, R₁₀, and R₁₀ each independently represent a hydrogen, an alkyl, an alkenyl, —(CH₂)ₘ-Rs or R₉ and R₁₀ taken together with the N atom to which they are attached complete a heterocycle having from 4 to 8 atoms in the ring structure; Rs represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R₁₀:) each independently represent a hydrogen, an alkyl or cycloalkyl, an alkenyl or cycloalkenyl, or alkynyl. Thus, the term “alkylamine” as used herein can refer to an amine group, as defined herein, having a substituted (as described hereinfor alkyl) or unsubstituted alkyl attached thereto, i.e., at least one of R₉ and R₁₀ is an alkyl group.

As used herein, the term “imide” can refer to —C(O)NR′R″, wherein R′ and R″ are each independently hydrogen, or a substituted or unsubstituted alkyl, cycloalkyl, alkenyl, alkynyl, aryl aralkyl, heterocyclyl or heterocyclylalkyl group as defined herein.

As used herein, the term “halogen” can refer to —F, —Cl, —Br or —I; the term “sulfhydryl” can refer to —SH; the term “hydroxyl” can refer to —OH; and the term “sulfonyl” can refer to —SO₂—.

The term “substituted” as used herein, refers to permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, for example 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups. As used herein in reference to an “R” group, the name used to describe said “R” group can be the chemical name prior to the removal of a hydrogen. For example, wherein “R” is described as an “alkane” can refer to an “alkyl” group.

Heteroatoms such as nitrogen can have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen can have hydrogen substituents and/or any permissible substituents of organic compounds described herein which satisfy the valencies of the heteroatoms.

In various aspects, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

As used herein, the term hydroxyalkyl can refer to a hydroxy terminated alkyl. For example, the hydroxyalkyl can be any hydroxyalkyl known in the art. In embodiments the hydroxyalkyl can be C₁-C₁₂ hydroxyalkyl. For example, the hydroxyalkyl can be hydroxymethyl, hydroxyethyl, hydroxypropyl, hydroxybutyl, hydroxypentyl, hydroxyhexyl, etc.

In embodiments, the terms “compound” and “composition” can be used interchangeably.

Thermal bonding, debonding (TBDB) often requires elevated temperatures and heat cycling. However, these temperature and heat cycling can lead to cracking or warping of the processed device. Described herein is a temporary bonding adhesive polymer and new monomer for producing the same.

Aspects of the disclosure are drawn towards a compound of Formula (I)

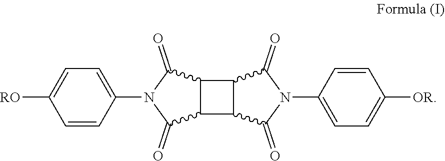

In embodiments, the compound of Formula (I) can be a monomer. In embodiments, the compound of Formula (I) can be a photo-active monomer.

In embodiments, R can be selected from the group consisting of H, hydroxyalkyl, or alkenyl. In embodiments, the hydroxyalkyl can be any hydroxyalkyl known in the art. For example, the hydroxyalkyl can be hydroxy C₁-C₁₂. For example, in non-limiting embodiments, the hydroxyalkyl can be hydroxymethyl, hydroxyethyl, hydroxypropyl, hydroxybutyl, or hydroxypentyl.

In embodiments, the hydroxyalkenyl can be alkenyl can be any alkenyl known in the art. For example, the alkenyl can be C₂-C₁₃ alkenyl.

As used herein, the term “photo-active” can refer to a compound or composition that can respond to light. As used herein, the terms “photo-active” and “photor-eactive” can be used interchangeably. For example, photo-reactive compounds and compositions described herein can undergo photocleavage. In embodiments, the butane ring of the compounds described herein can be cleaved. For example, the compounds and compounds can undergo photocleavage at a wavelength of about 300 nm or less. For example, the compounds and compositions described herein can undergo photocleavage at about 254 nm.

In embodiments, the compound described herein is:

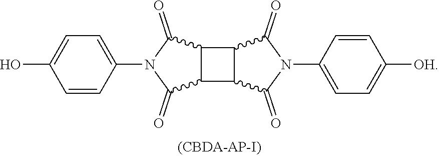 [SMILES: O=C1C2C(C(=O)N1c1ccc(O)cc1)C1C(=O)N(c3ccc(O)cc3)C(=O)C21]

In embodiments, the compound described herein is:

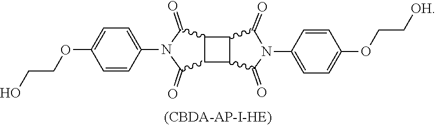

In embodiments, the compound described herein is:

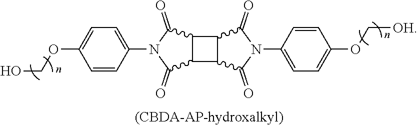

In embodiments, wherein n is 1 to 12. Non-limiting examples include:

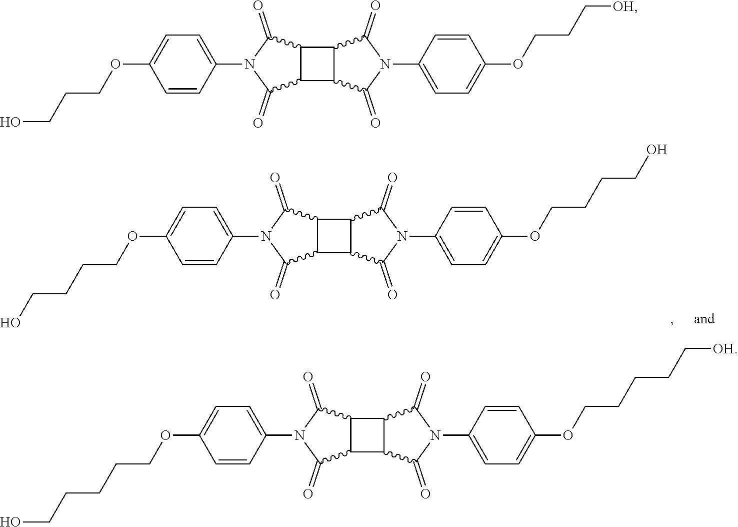 [SMILES: O=C1C2C(C(=O)N1c1ccc(OCCCO)cc1)C1C(=O)N(c3ccc(OCCCO)cc3)C(=O)C21 ; O=C1C2C(C(=O)N1c1ccc(OCCCCO)cc1)C1C(=O)N(c3ccc(OCCCCO)cc3)C(=O)C21 ; O=C1C2C(C(=O)N1c1ccc(OCCCCCO)cc1)C1C(=O)N(c3ccc(OCCCCCO)cc3)C(=O)C21]

In embodiments, the installation of the hydroxyalkyl functionality can be completed through a cyclic carbonate ring opening reaction, or through the reaction between a halogen functionalized alcohol. A mild base capable of deprotonating the phenol while leaving the hydroxyalkyl group protonated encourages the SN2 reaction between the resulting alkoxide and halogenated carbon. Through this method an alkyl spacer of length C₁-C₁₂ can be installed on the CBDA-AP-I monomer. The Scheme 6 shows this reaction where X is any halogen and R is any carbon space length C₁-C₁₂

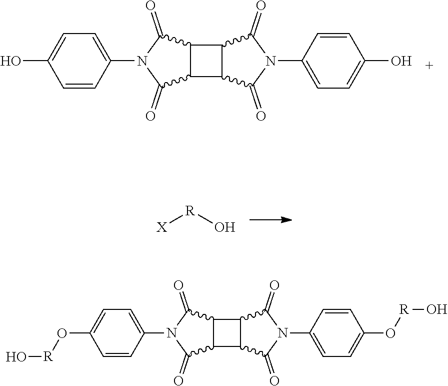

Scheme 6: shows the reaction where X is any halogen and R is any carbon space length C₁-C₁₂.

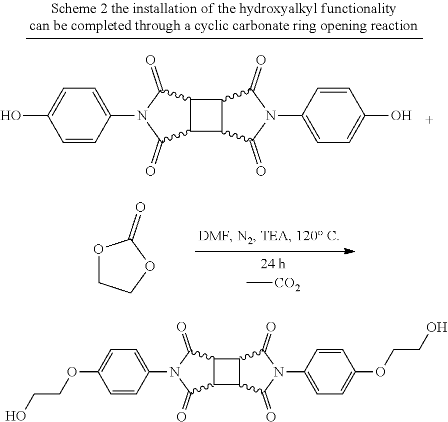

In embodiments, the compound described herein is:

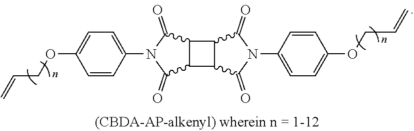

In embodiments, the installation of the alkenyl group can occur via a Micheal reaction through conditions known in the art. For example, the Michael reaction can occur under mild basic conditions (K₂CO₃) between the deprotonated CBDA-AP-I and a Michael acceptor such as a halogenated (X) alkene to produce an alkene functionalized CBDA-AP-I. For example, see Scheme 7.

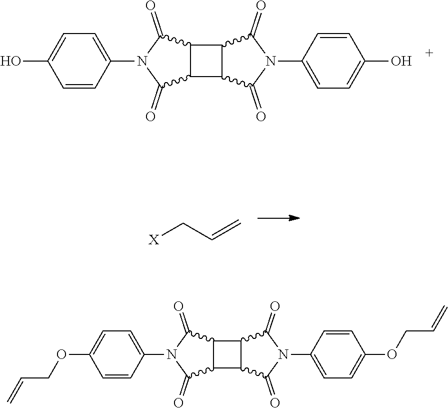

Scheme 7 shows the installation of an alkenyl group.

Aspects of the invention are drawn towards a method of producing a photoactive monomer. In embodiments, the method comprises reacting 1,2,3,4-cyclobutane-tetracarboxylic dianhydride (CBDA) with 4-aminophenol (AP) in at least a 1:2 molar ratio, thereby generating

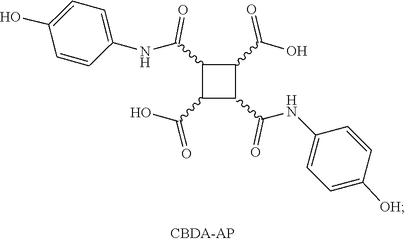 [SMILES: O=C(O)C1C(C(=O)Nc2ccc(O)cc2)C(C(=O)O)C1C(=O)Nc1ccc(O)cc1]

and subjecting the CBDA-AP to imidization, thereby producing

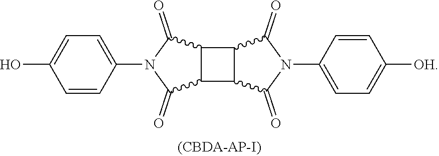 [SMILES: O=C1C2C(C(=O)N1c1ccc(O)cc1)C1C(=O)N(c3ccc(O)cc3)C(=O)C21]

In embodiments, the imidization is selected from the group consisting of thermal imidization or chemical imidization. As used herein, the thermal imidization can comprise increased heat or increased heat and reduced pressure. As used herein, the chemical imidization can comprise reactions with reagents or catalysts as known in the art.

Aspects of the invention are drawn towards a polymer comprising the monomer of Formula (I)

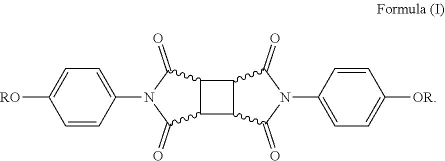

In embodiments, R is selected from the group consisting of of H, alkenyl, or hydroxyalkyl. For example, the hydroxyalkyl can be hydroxymethyl, hydroxyethyl, hydroxypropyl, hydroxybutyl, hydroxypentyl, etc. The hydroxyalkyl group can be installed on the CBDA-AP-I monomer through a ring opening polymerization (see, e.g., Scheme. 2 reaction with ethylene carbonate), a Micheal reaction, or through any ester-forming reaction known in the art, e.g., Williamson ether synthesis. In embodiments, the polymer can comprise the monomer of Formula (I). In embodiments, the polymer can comprise the monomer of Formula (I) and at least one co-monomer. In embodiments, R is selected from the group consisting of H or hydroxyethyl.

In embodiments, the polymer comprises the monomer of Formula (I) and the polymer can be:

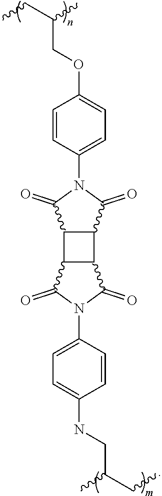

In embodiments, n and m can be any number between 0 to 100.

In embodiments, the alkene functionalized CBDA-AP-I can be reacted with a radical initiator to produce a crosslinked network (see, e.g., Scheme 8) that can be deconstructed with 254 nm light. Likewise, this vinyl ether functionalized monomer can be coreacted with bismaleimde monomers to produce a crosslinked structure.

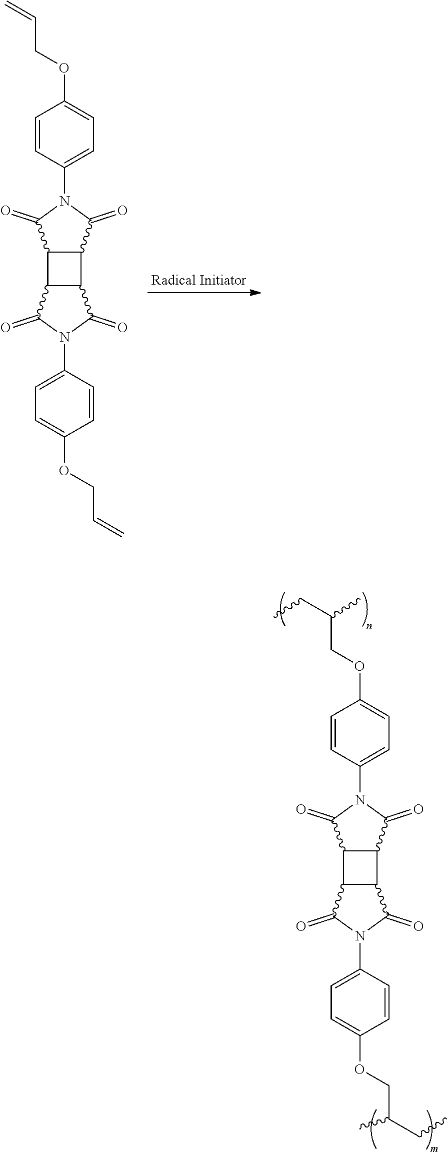

Scheme 8 shows a non-limiting, exemplary polymerization described herein. This reaction can occur with conditions known in the art.

As used herein, the term “monomer” can refer to any discreet chemical compound of any molecular weight. As used herein, the term “co-monomer” can refer to a polymerizable precursor to a co-polymer in addition to a first monomer. For example, the reaction with co-monomers can produce a copolymer. The term “copolymer” as used herein, can refer to a single polymeric material that is comprised of two or more different monomers. The copolymer can be of any form, such as gradient, random, block, graft, etc. The copolymers can have any end-group.

As used herein, the term “polymer” refers to a molecule composed of repeating structural units typically connected by covalent chemical bonds. The term “polymer” is also meant to include the terms copolymer and oligomers. In certain embodiments, a polymer comprises a backbone (i.e., the chemical connectivity that defines the central chain of the polymer, including chemical linkages among the various polymerized monomeric units) and a side chain (i.e., the chemical connectivity that extends away from the backbone).

As used herein, the term “polymerization” can refer to at least one reaction that consumes at least one functional group in a monomeric molecule (or monomer), oligomeric molecule (or oligomer) or polymeric molecule (or polymer), to create at least one chemical linkage between at least two distinct molecules (e.g., intermolecular bond), at least one chemical linkage within the same molecule (e.g., intramolecular bond), or any combinations thereof. A polymerization reaction can consume between about 0% and about 100% of the at least one functional group available in the system. In certain embodiments, polymerization or crosslinking of at least one functional group results in about 100% consumption of the at least one functional group. In other embodiments, polymerization or crosslinking of at least one functional group results in less than about 100% consumption of at least one functional group.

As used herein, the term “reaction condition” can refer to a physical treatment, chemical reagent, or combination thereof, which is required or optionally required to promote a reaction. Non-limiting examples of reaction conditions are electromagnetic radiation (such as, but not limited to, visible light and UV light), heat, a catalyst, a chemical reagent (such as, but not limited to, an acid, base, electrophile or nucleophile), and a buffer. Reaction conditions as described can be altered by one of ordinary skill in the art as known in the art.

In embodiments, the polymers described herein are photocleavable. As used herein, the term “photocleavable” can refer to a compound or moiety that can be cleaved in response to light. For example, in non-limiting, exemplary embodiments, the butane ring of the compounds described herein are photocleavable when exposed to light that is about 300 nm or less.

In embodiments, the polymers described herein can be temporary bonding adhesive polymers. As used herein, the term “temporary bonding adhesive polymers” can refer to a structural adhesive. In embodiments, the structural adhesive can withstand a manufacturing process but can then be debonded after processing.

In embodiments, the polymer described herein can be a polyurethane, a polyester, a polyketone, a polysulfone, polysiloxone, or a co-polymer thereof. These polymers can be synthesized through polymerization methods known in the art by one of ordinary skill in the art.

In embodiments, the polymer described herein can comprise a repeating unit of

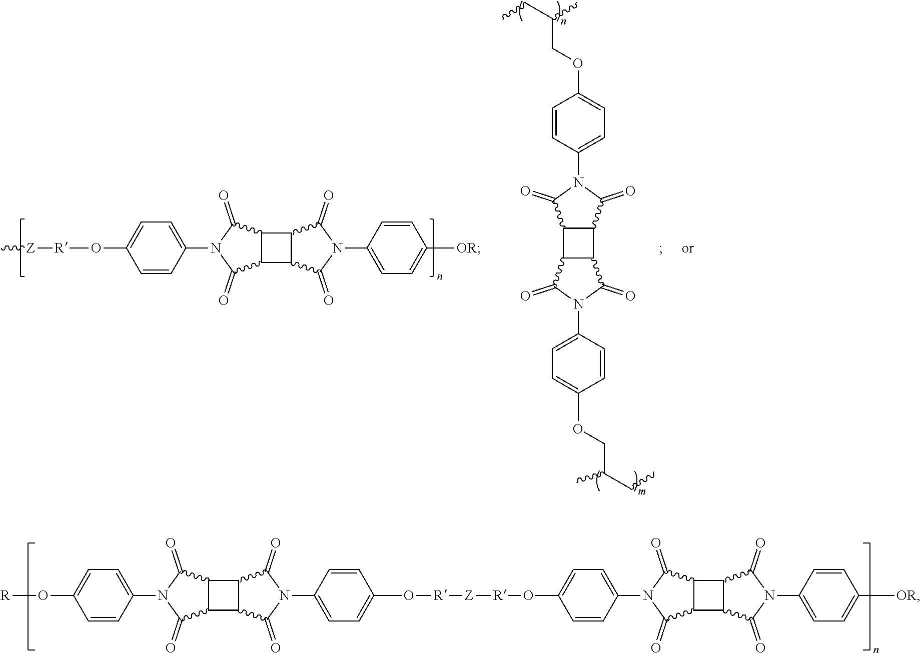

wherein, R is selected from the group consisting of H or hydroxyalkyl; R′ is absent when R is H, or R′ is-O-alkyl when R is hydroxyalkyl; and Z is a polyurethane, polyester, polysulfone, polyketone, or a monomer thereof.

For example, if R is hydroxyethyl, R′ will be-O-ethyl. For example, the structure would be:

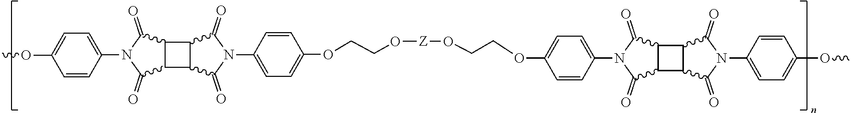

In embodiments, the notation [symbol] can refer to a bond where the stereospecificity is not defined. In embodiments, [symbol] can refer to a mixture of isomers. In embodiments, [symbol] can refer to the molecule extending beyond the indicated bond. For example, see chem.libretexts.org/Bookshelves/Organic_Chemistry/Map % 3A_Organic_Chemistry_(Smith)/24% 3A_Synthetic_Polymers/24.01%3A_Introduction and chem.ucla.edu/˜harding/IGOC/W/wavy_line.html.

In embodiments, the polymer described herein can comprise about 1 mol. % to about 99 mol. % of the structure of Formula I. In embodiments, the polymer comprises the monomer of Formula (I) and at least one additional co-monomer. In embodiments, the at least one additional co-monomer can be selected from the group consisting of an isocyanate terminated monomer or polymer; a di-carboxylic acid; a silicone containing monomer,

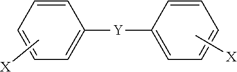

wherein X═Cl or F, and Y=SO₂ or C═O; or
a combination thereof. For example, the co-monomer of

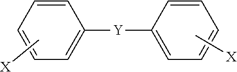

can be selected from the group consisting of:

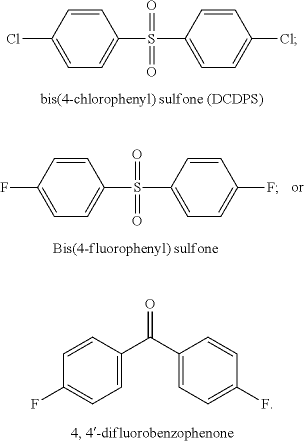 [SMILES: O=S(=O)(c1ccc(Cl)cc1)c1ccc(Cl)cc1 ; O=S(=O)(c1ccc(F)cc1)c1ccc(F)cc1 ; O=C(c1ccc(F)cc1)c1ccc(F)cc1]

In embodiments, the co-monomer can be

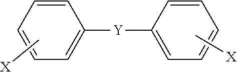

wherein X═Cl or F, and
Y=SO₂ or C═O and the polymer can further comprise a diphenol co-monomer. In embodiments, the diphenol can be any diphenol known in the art. For example, the diphenol can be

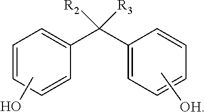

In embodiments, R₂ and R₃ can be independently selected from the group consisting of H and alkyl. For example, the diphenol can be any bisphenol known in the art. For example, the bisphenol can be

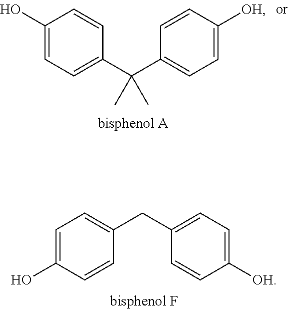 [SMILES: CC(C)(c1ccc(O)cc1)c1ccc(O)cc1 ; Oc1ccc(Cc2ccc(O)cc2)cc1]

In embodiments, the di-carboxylic acid can be any di-carboxylic acid known in the art. For example, the di-carboxylic acid can be

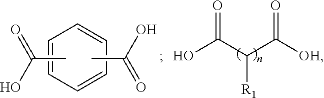

wherein R₁ is selected from the group consisting of H, alkyl, or aryl;

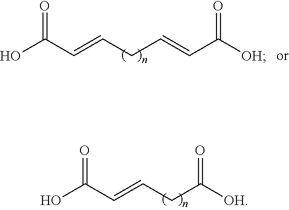

In embodiments, the co-monomer can be a siloxane monomer as known in the art.

In embodiments, the polymer can be a CBDA-AP-I/PDMS co-monomer. For example, the polymer can be:

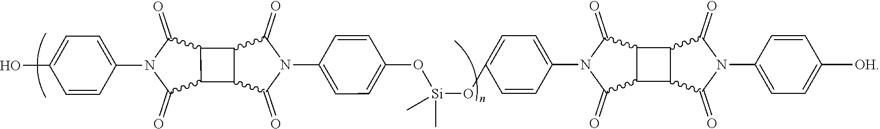

In embodiments, the co-monomer can be an isocyanate terminated monomer or polymer. For example, the reaction between the isocyanate terminated monomer and the monomer of Formula (I) can produce new polyurethanes. In some embodiments, the isocyanate terminated polymer can comprise a polyester backbone. For example, the polyester backbone can be polyethylene glycol, polypropylene glycol, or any polyester known in the art.

In embodiments, the isocyanate terminated monomer or polymer can be any isocyanate terminated monomer or polymer known in the art. For example, the isocyanate polymer can be selected from the group consisting of:

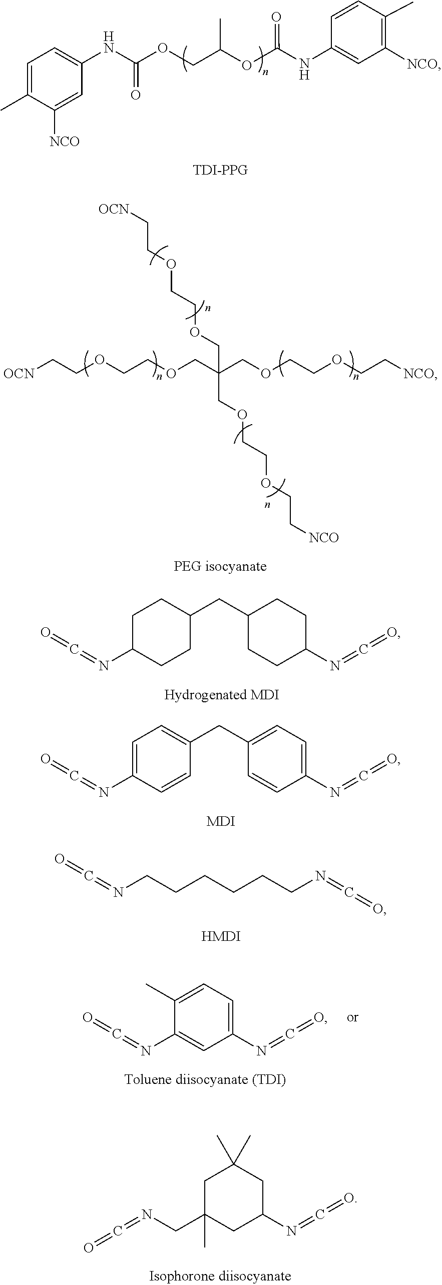

In embodiments, the monomer of Formula (I) and the isocyanate terminated monomer or polymer are present in about a 99:1 to about 99:1 molar ratio. For example, the monomer of Formula (I) and the isocyanate terminated monomer or polymer can be in a ratio of about 95:5, about 90:10, about 85:15, about 80:20, about 75:25, about 70:30, about 65:35, about 60:40, about 55:45, about 50:50, about 40:60, about 30:70, about 25:75, about 20:80, about 15:85, about 10:90, or about 5:95.

In embodiments, the polymer can be:

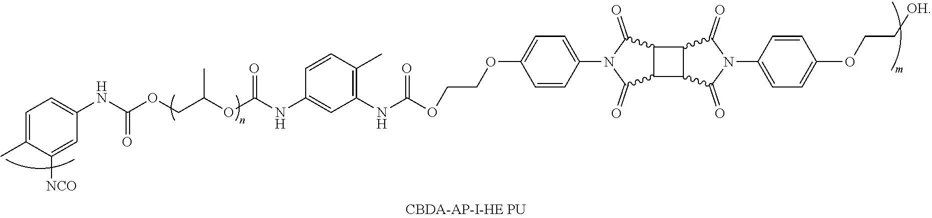

In embodiments, the polyurethanes described herein can comprise a molecular weight of less than about Mn=3,000 g/mol to greater than about Mn=250,000 g/mol.

In embodiments, the monomer of Formula (I) can be reacted with co-monomers to form polysulfone copolymers. For example, the co-monomers can be a diphenol and

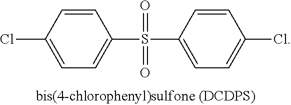 [SMILES: O=S(=O)(c1ccc(Cl)cc1)c1ccc(Cl)cc1]

In some embodiments, the diphenol is a slight molar excess of DCDPS. As used herein, the term “slight molar excess” can refer to a ratio of about 1:1.05 to about 1:1.1. However, this ratio can be determined by one of ordinary skill in the art based upon the reaction.

In embodiments, the diphenol can be any diphenol known in the art. In embodiments, the diphenol is

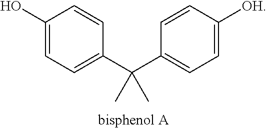 [SMILES: CC(C)(c1ccc(O)cc1)c1ccc(O)cc1]

In embodiments, the polymer comprises the monomer of Formula (I) in about 1 mol. % to about 100 mol. % of the diphenol. For example, the polymers can comprise the monomer of Formula (I) in about 1 mol. %, about 2 mol. %, about 3 mol. %, about 4 mol. %, about 5 mol. %, about 6 mol. %, about 7 mol. %, about 8 mol. %, about 9 mol. %, about 10 mol. %, about 12.5 mol. %, about 15 mol. %, about 17.5 mol. %, about 20 mol. %, about 22.5 mol. %, about 25 mol. %, about 27.5 mol. %, about 30 mol. %, about 32.5 mol. %, about 35 mol. %, about 37.5 mol. %, about 40 mol. %, about 42.5 mol. %, about 45 mol. %, about 47.5 mol. %, about 50 mol. %, about 55 mol. %, about 60 mol. %, about 65 mol. %, about 70 mol. %, about 75 mol. %, about 80 mol. %, about 85 mol. %, about 90 mol. %, about 95 mol. %, or about 99 mol. % of the diphenol. For example, the monomer of Formula (I) is present in about 2.5 mol. % to about 50 mol. % of the diphenol.

In embodiments, polymers described herein can comprise a cyclobutane bisimide monomer described herein in less than about 1 mol % cyclobutane bisimide monomer, about 1 mol % cyclobutane bisimide monomer, about 2.5 mol % cyclobutane bisimide monomer, about 5 mol % cyclobutane bisimide monomer, about 10 mol % cyclobutane bisimide monomer, about 15 mol % cyclobutane bisimide monomer, about 20 mol % cyclobutane bisimide monomer, about 25 mol % cyclobutane bisimide monomer, about 30 mol % cyclobutane bisimide monomer, about 35 mol % cyclobutane bisimide monomer, about 40 mol % cyclobutane bisimide monomer, about 45 mol % cyclobutane bisimide monomer, about 50 mol % cyclobutane bisimide monomer, about 55 mol % cyclobutane bisimide monomer, about 60 mol % cyclobutane bisimide monomer, about 65 mol % cyclobutane bisimide monomer, about 70 mol % cyclobutane bisimide monomer, about 75 mol % cyclobutane bisimide monomer, about 80 mol % cyclobutane bisimide monomer, about 85 mol % cyclobutane bisimide monomer, about 90 mol % cyclobutane bisimide monomer, about 95 mol % cyclobutane bisimide monomer, greater than about 95 mol % cyclobutane bisimide monomer, or 100 mol % cyclobutane bisimide monomer.

In embodiments, the polymer described herein can be a co-polymer. For example, the polymer described herein can be

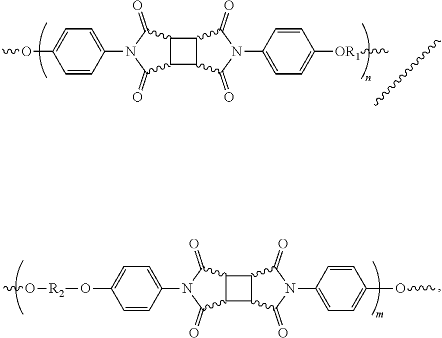

wherein R₁ and R₂ is any combination of a polyurethane, polysulfone, polyketone, polyester, polysiloxane, or a monomer thereof. In embodiments, n=0.01-0.99; and m=0.99-0.01.

In embodiments, the polymer can be

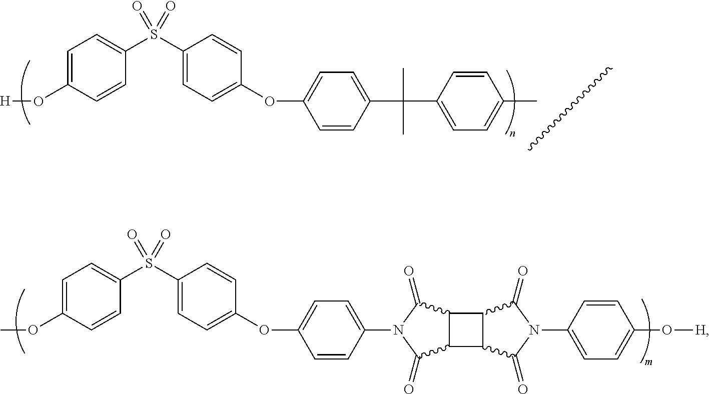

wherein n=0.01-0.99; and m=0.99-0.01

Aspects of the invention are drawn towards a method of producing a cyclobutane bisimide polyurethane (PU), the method comprising reacting CBDA-AP-I-HE with an isocyanate terminated monomer or polymer, thereby producing a cyclobutane bisimide polyurethane (PU). In embodiments, the method further comprises reacting the CBDA-AP-I-HE and the isocyanate terminated monomer or polymer with an additional diol. In embodiments, the isocyanate terminated monomer or polymer is selected from the group consisting of:

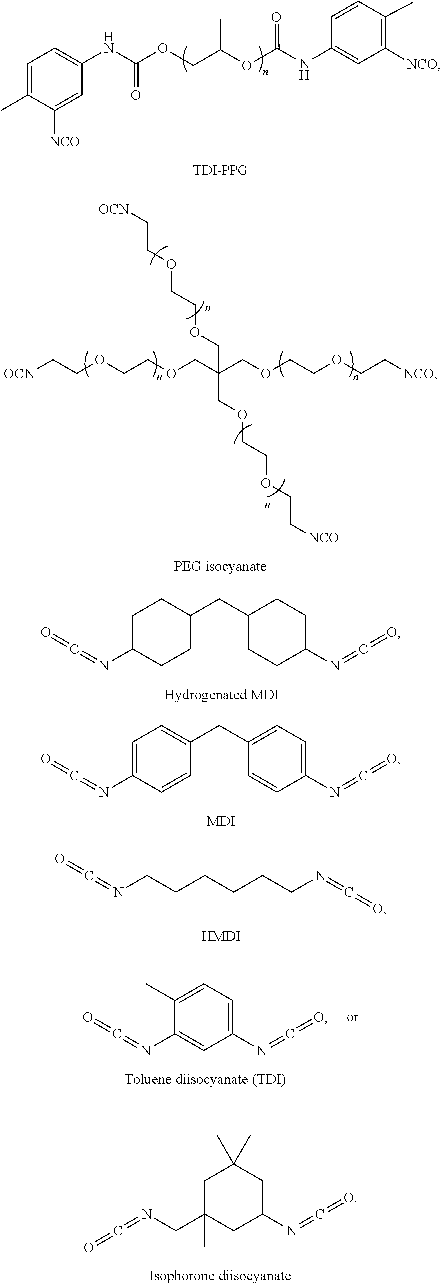

In embodiments, the cyclobutane bisimide polyurethane is:

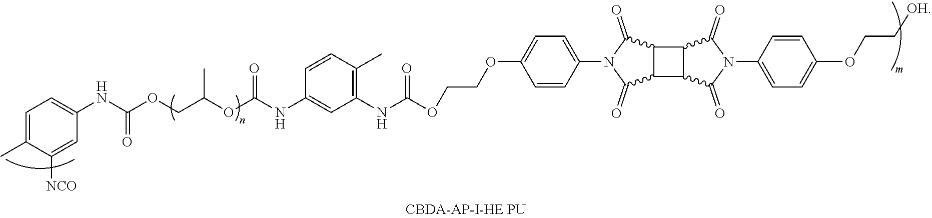

Aspects of the invention are drawn towards a cyclobutane bisimide polyurethane produced by the method described herein.

Aspects of the invention are drawn towards a method of producing a cyclobutane bisimide polysulfone (PSU) the method comprising reacting CBDA-AP-I with a diphenol and bis(4-chlorophenyl) sulfone (DCDPS), thereby producing a cyclobutane bisimide polysulfone (PSU). In embodiments, the diphenol is bisphenol A. In embodiments, CBDA-AP-I is present in about 1% to about 100% mol. %. In embodiments, CBDA-AP-I is present in about 2.5 mol. % to about 50 mol. %. In embodiments, the cyclobutane bisimide polysulfone (PSU) is

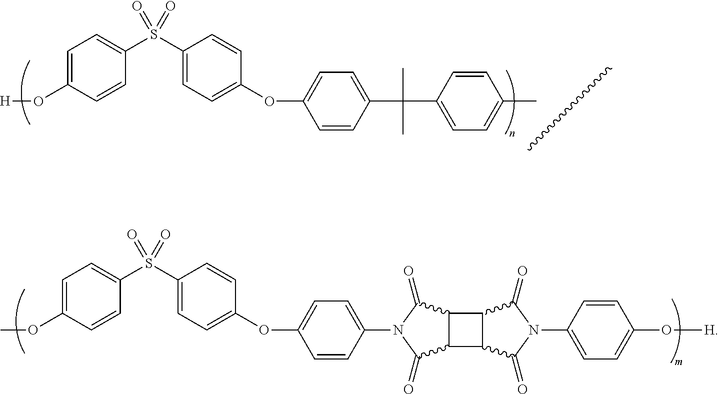

In embodiments, the copolymers described herein can be in a ratio of n−m=1.

In embodiments, the polymers described herein can comprise a range of molecular weights than can be achieved by one of ordinary skill in the art by methods known in the art. For example, the polymers described herein can comprise a molecular weight of about Mn=1000 to about Mₙ=250,000. In some embodiments, the polymers described herein can be less than about Mₙ=1000 and greater than about Mₙ=250,000.

Aspects of the invention are drawn towards a cyclobutane bisimide polysulfone (PSU) produced by the method described herein. Aspects of the invention are drawn towards a cyclobutane bisimide polyether produced by the methods described herein. Aspects of the invention are drawn towards a cyclobutane bisimide polyketone produced by the methods described herein. Aspects of the invention are drawn towards a cyclobutane bisimide copolymer produced by the methods described herein. The methods described herein can be combined with methods known in the art to produce variations of compositions described herein.

Aspects of the invention are drawn towards a photocleavable, temporary bonding adhesive polymer comprising the monomer described herein.

Aspects of the invention are drawn towards a method of temporarily bonding a support substrate to a semiconductor wafer for processing. In embodiments, the method comprises obtaining a device wafer and a support substrate; applying the photocleavable adhesive polymer described herein to the surface of the support substrate, thereby forming an adhesive layer; adhering the device wafer to the support substrate by placing it on top of the adhesive layer; processing the device wafer; and irradiating the adhesive layer with 254 nm light, thereby debonding the support substrate from the device wafer. In embodiments, the support substate comprises quartz or glass. In embodiments, the polymer is applied via spincoating.

Aspects of the invention are drawn towards a photodegradable polymer. In embodiments, the polymer can be used in sustainable initiatives. For example, the polymer can be photo depolymerized, purified, and then repolymerized with light. In embodiments, the photodegradable polymer can be used in medical devices or prosthetics. In embodiments, the polymer described herein can be used in materials for biohazardous applications. For example, the polymer described herein can be used in laboratory materials that can be depolymerized after use.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### A New Cyclobutene Bisimide Monomer for Photo-Cleavable Polyurethane Synthesis

Innovations in the semiconductor industry require the development of new processing methods to accommodate advanced development techniques. Temporary bond-debond (TBDB) technology represents a processing modality that requires constant innovation. Previous work illustrates the use of 1,2,3,4-cyclobutane-tetracarboxylic dianhydride (CBDA) as a pathway for photo-cleavable TBDB technology that utilizes a poly(dimethyl siloxane) backbone. This work presents the synthesis of a new cyclobutane bisimide monomer that boasts either bisphenol or bishydroxy reactivity. The reactivity of 4-aminophenol allowed for selective addition of the aryl amine onto CBDA, producing an amic acid precursor capable of chemical and thermal imidization. FTIR and ¹H NMR spectroscopy, coupled with thermogravimetric analysis, fully characterized the synthetic pathway for this new monomer. Subsequent polymerization with a toluenediisocyante terminated poly(propylene glycol) monomer yielded a new cyclobutane functionalized polyurethane for TBDB applications. Thermomechanical analysis revealed a 27° C. increase in flow temperature and 20% increase in strain after irradiation with 254 nm light. Likewise, ¹H NMR spectroscopy characterized the formation of maleimide protons resulting from cyclobutane bisimide ring cleavage after light exposure.

### INTRODUCTION

As competitive innovation continues in the semiconductor industry, the development of new materials capable of supporting technically challenging patterning processes continue to progress. These materials must meet diverse thermomechanical and optical requirements for multi-step fabrication processes, with an emphasis on thermal expansion coefficients, thermal stability, off-gassing, and photo-activity.¹ One such use case is the temporary bonding of a substrate onto a supporting layer during the fabrication process, known as temporary bonding and debonding (TBDB).²⁻⁴ This involves an initial bonding process between a glass substrate and the wafer that subsequently undergoes development. The wafer must then be released from the substrate through a debond process that requires a release mechanism and washing of the bonding material off the wafer.³ A facile release mechanism of the TBDB process is required to ensure minimal damage to the wafer surface, as thermal debonding of the adhesive often demands elevated temperatures and heat cycling that leads to warpage or cracking of the processed device.⁵⁻⁶ For this reason, a departure from the traditional thermal process is desired.

Light-based debonding techniques have received considerable attention due to lower processing costs and higher control.^{2.7-9} Research has indicated cyclobutane bisimide functionalities for bond/debond and patterning applications.⁹⁻¹⁰ Among structural adhesives, the imide functionality displays advanced thermal stability along with low static permittivity, chemical stability, and dielectric properties that result in their prolific use in the semiconductor sector.¹¹ There is research into photo-degradable polymers for application to semiconductor patterning and processing.¹²⁻¹⁸ These systems can be useful in various steps of the semiconductor packaging process due to their photo-crosslinking or photo-degradation potential.

Photo-responsive cyclobutane chemistries are researched for a variety of applications including thymine, stilbene, cinnamate, and malcimide derivatives for use in photo-reversible adhesives, self-healing polymers, liquid crystals, and light harnessing. ¹⁹⁻²¹ Additionally, they possess dimerization wavelengths between 350 and 270 nm and photocleavage excitations below 300 nm.¹⁹⁻²² This broad range of photoactivity allows for careful selection of cyclobutane chemistry to match a targeted use scenario, i.e., selecting a high-energy reversal wavelength ˜254 nm can allow for exposure of the system to ambient light, since that wavelength displays low intensity at ambient conditions. Thus, the selection of cyclobutane bisimide functionality enables photo-reversibility while requiring high intensities of low wavelength light for photo-cleavage. This is advantageous as it allows for orthogonal light-based processes to occur concurrently without the possibility of unwanted cyclobutane ring reversal subsequently allowing for facile integration into present packaging modalities.

A CBDA technology that is readily applied to a wide variety of polymeric systems remains desired.⁹ Thus, the synthesis of a cyclobutane bisimide-containing monomer capable of photo-degradation is described herein. The greater reactivity of the nucleophilic aryl amine of 4-aminophenol enabled a selective addition to CBDA, producing a cyclobutane-containing amic acid with bisphenol functionality. Subsequent imidization resulted in the cyclobutane-bisimide moiety with bisphenol reactivity. Furthermore, reacting this imide with ethylene carbonate resulted in the installation of hydroxy ethyl groups allowing for the selection of phenol or hydroxyl reactivity. This expands the application space for this material, broadening the scope of this monomer to both fully aromatic and aliphatic systems. Subsequent polymerization of this monomer with diisocyanates resulted in the synthesis of a new, cyclobutane bisimide-containing polyurethane capable of on-demand photo-degradation.

### Materials and Methods:

### Materials

Cyclobutane-1,2,3,4-tetracarboxylic diahnhydride (CBDA) was graciously provided by Jayhawk Fine ChemicalsR and used as received. 4-Aminophenol (>98%), 3-aminophenol (>98%), deuterated dimthylsulfoxide-d6 (99.95%), ethylene carbonate (98%), triethylamine (TEA) (>99.5%), poly(propylene glycol), toluene 2,4-diisocyanate terminated Mₙ=2,300 g/mol (PPG-TDI), and 1,4-diazabicyclo[2.2.2]octane (dabco) were purchased from SigmaAldrich and used as received. N,N-dimethylformamide (DMF) (>99.5%), ethyl acetate (>99.5% ACS grade), toluene (>99%), acetone (>99.5%), and 1,2-dichlorobenzene (>99%) were purchased from VWR and used as received.

### Instruments

¹H and ¹³C nuclear magnetic resonance (NMR) spectroscopy utilized a Bruker Avance NEO 500 MHz spectrometer functioning at 500.15 MHz and 23° C. (solution concentration of 10 mg mL 1). A ThermoFisher Scientific Nicolet iS10 FTIR spectrometer, with a diamond cell at 25° C., identified key stretches for synthesized monomers and polymers. A TA Instruments TGA 5500 facilitated thermogravimetric analysis (TGA) by utilizing a heating rate of 10° C. min 1 from 25 to 600° C. with a steady nitrogen purge. The T_{d.5}%, or temperature where 5% of the original sample mass was lost, served as an indicator for sample thermal stability. A TA Instruments DSC 2500 with heat/cool/heat cycles of 10° C. min 1 provided differential scanning calorimetry data where the sample was under a nitrogen environment throughout the experiment. DSC provided the glass transition temperatures (TgS) from the midpoint of the endothermic transition in the second heat. Polyurethane films were casted out of DMF at a concentration of 100 mg/mL onto a Teflon® petri dish at 50° C. A TA Instruments DMA Q800 with a temperature ramp of 3° C. min 1 from −90° C. to 180° C. at 1 Hz provided a T_{g} taken from the peak in the tan delta. Liquid nitrogen cooling permitted the cryogenic temperatures required to observe the thermal transitions of the polyurethane films. A Mineralight 254 nm 15 w lamp equipped with a 254 nm filter enabled consistent irradiation of samples at an intensity of 4.8 mW/cm². A Waters ARC HPLC size exclusion chromatography system operating at a flow rate of 1 mL/min at 5 mg/mL and temperature of 50° C. enabled molecular weight determination for polymeric samples. Samples were run in 0.05 M LiBr DMF and passed through a 0.45 micrometer filter before injecting. A Waters dRI detector monitored concentration change and final molecular weight was determined against a polystyrene calibration curve. An Instron 68TM-5 with a 5 kN load cell enabled tensile testing of PU films at a rate of 5 mm/min.

### Chemical and Thermal Synthesis of CBDA-AP-I:

CBDA (10.00 g, 51.0 mmol), 4-aminophenol (11.13 g, 102.0 mmol), 100 mL of toluene and 500 mL of 1,2-dichlorobenzene were added to a 3-neck round bottomed flask equipped with a magnetic stir bar, nitrogen inlet, reflux condenser, and Dean-Stark apparatus. Reagents were allowed to mix and subsequently heated to 180° C. The reaction was allowed to progress for 18 h and remained heterogenous throughout. The toluene served to remove any water produced throughout the imidization reaction through an azeotrope and the Dean-Stark apparatus was emptied as needed. The precipitate was filtered using a fritted funnel and allowed to dry overnight then recrystallized in DMF. The final isolated yield was >90%. ¹H NMR (DMSO-d6, δ, FIG. 5) 9.77 (s, 2H), 7.2 (d, 4H, J=8.5 Hz), 6.87 (d, 4H, J=8.5 Hz), 3.62 (s, 4H).

### Synthesis of CBDA-AP-I-HE

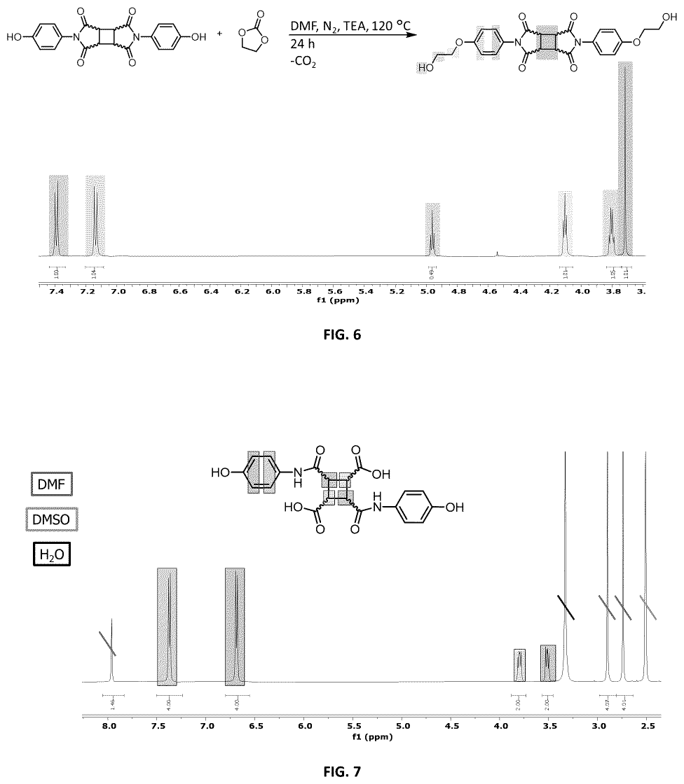

CBDA-AP-I (5 g, 13.21 mmol), ethylene carbonate (3.49 g, 39.64 mmol) and TEA (4.01 g, 39.64 mmol) were added to a 2-necked 1000 mL round bottomed flask equipped with a nitrogen inlet, magnetic stir bar, and reflux condenser. 500 mL of DMF was added and heated to 120° C. under nitrogen flow. The reaction was allowed to proceed for 24 h before precipitating into ethyl acetate. The precipitate was collected with a fritted funnel and dried under reduced pressure for 12 h at 120° C. 1IH NMR (DMSO-d6, δ, FIG. 6) 7.39 (d, 4H), 7.15 (d, 4H), 4.97 (s, 2H), 4.09 (t, 4H), 3.81 (t, 4H), 3.75 (s, 4H).

### Synthesis of CBDA-AP-I-HE PU

Dry DMF was added to a 200 mL 2-neck round bottomed flask equipped with a magnetic stir bar and purged with nitrogen. CBDA-AP-I-HE (5 g, 10.7 mmol), PPG-TDI (24.65 g, 10.7 mmol), and dabco (0.36 g, 3.2 mmol) were added to the flask and the reaction was heated to 80° C. The reaction was allowed to progress for 12 h and then precipitated into acetone. The collected product was dried under reduced pressure at 120° C. for 12 h. CBDA-AP-I PU films were cast out of DMF at a 15 wt. % PU loading. The PU solution was placed in a Teflon® dish and allowed to air dry at 40° C. for 24 h. The film was then heated to 100° C. under reduced pressure for 12 h to ensure removal of DMF.

### Results and Discussion

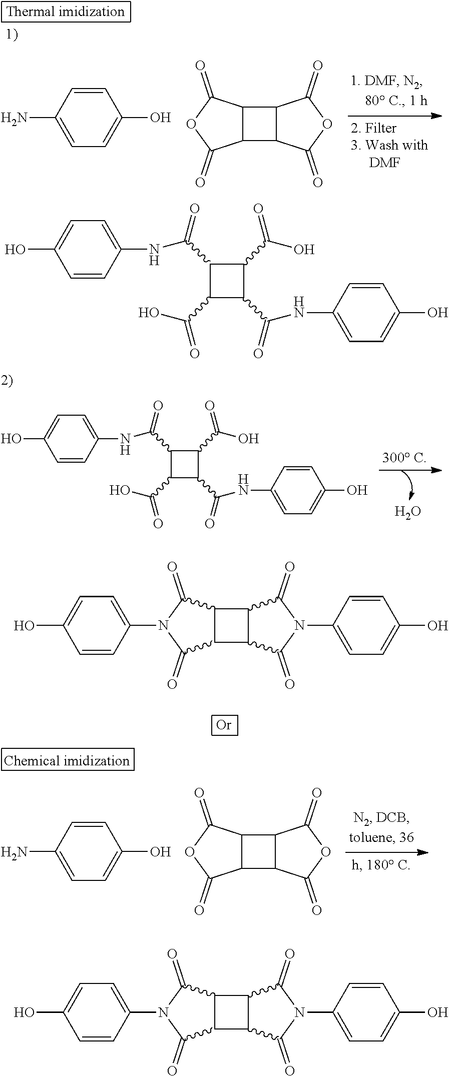

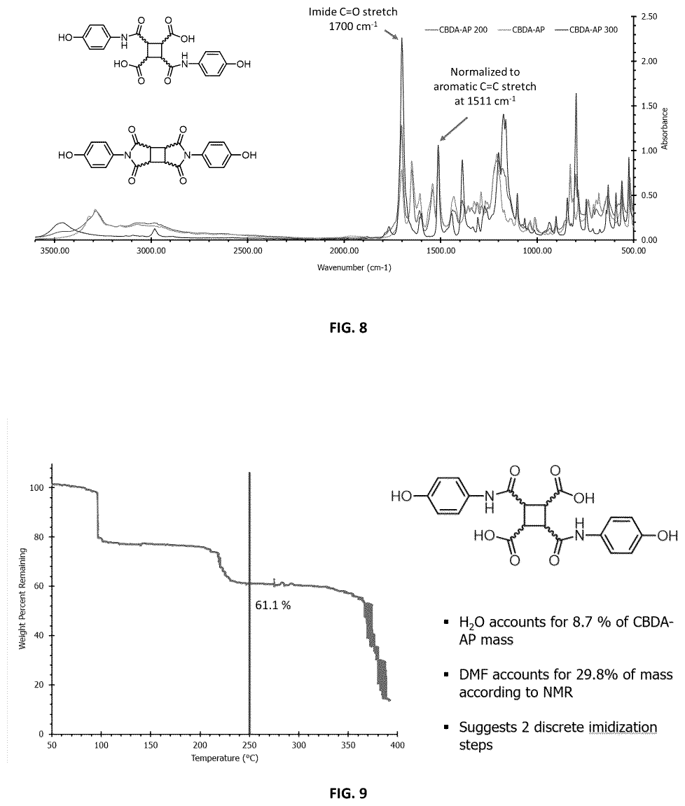

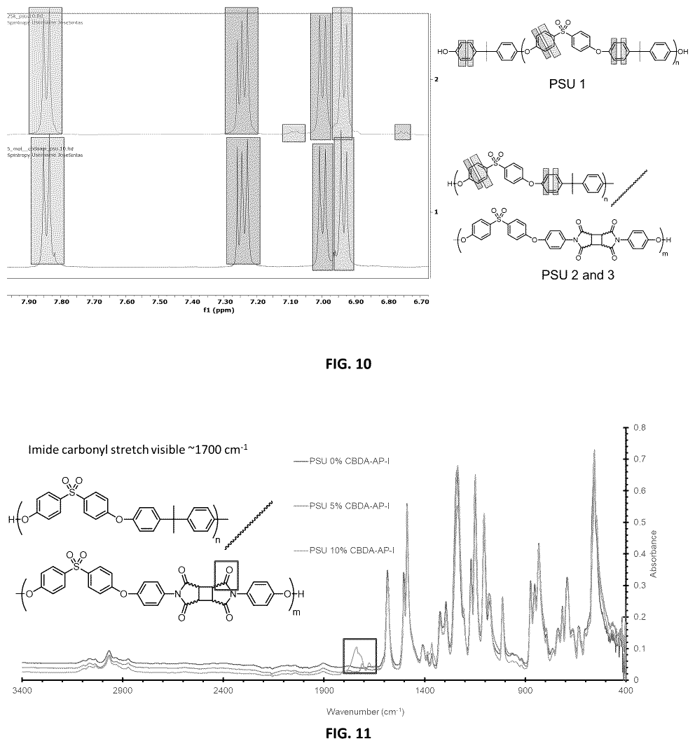

4-aminophenol (AP) possesses both aryl amine and phenol reactivity arranged in a para configuration, however the more reactive aryl amine directed the preferential formation of an amic acid precursor. This preferential addition, confirmed with ¹H NMR analysis, provided bisphenol reactivity after either chemical or thermal imidization. The thermal imidization route utilized isolation of the phenol functionalized amic acid precursor followed by a thermal imidization similar to conventional imidization techniques, shown in Scheme 1. It is interesting to note, however, that thermal imidization occurred at two distinct temperatures, 96 and 230° C., as opposed to a wide imidization window observed for polymeric imidization. FTIR, TGA, and NMR analysis of the amic acid, partially imidized, and fully imidized CBDA-AP-I compounds confirmed this phenomenon (FIGS. 7-9). Without wishing to be bound by theory, this can be due to the small molecule nature of this compound, as opposed to a polymeric one, where various steric and chemical environments exist. While effective at achieving high conversions, the thermal imidization route resulted in discoloration of CBDA-AP-I due to the degradation of residual DMF bound to the amic acid precursor. This discoloration can block 254 nm light, causing a decrease in photo-cleavage efficiency. Thus, a chemical imidization process that reduced coloration of CBDA-AP-I was developed, shown in Scheme 1. A one-pot reaction that leveraged a DCB and toluene mixture proved effective at achieving high purity, uncolored CBDA-AP-I. Toluene enabled the effective removal of the water condensate produced by the imide ring-closing reaction through azeotrope formation, promoting imidization.

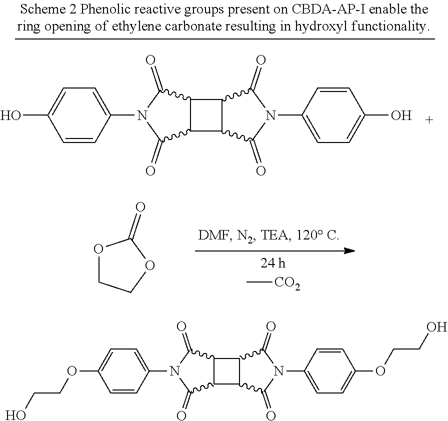

Imidization of CBDA-AP allows for a thermally stable bisphenol that readily incorporates into polymer backbones including polyesters, polycarbonates, polyurethanes, and polysulfones.²³⁻²⁴ While versatile, phenolic linkages display reduced thermal stability within certain chemistries. Additionally, the rigid imide and aromatic structures comprising CBDA-AP-I hinder solubility, requiring temperatures above 80° C. and high-boiling polar aprotic solvents for full dissolution. The installation of hydroxyethyl functionality to phenols allows for greater reactivity and solubility which offered an effective remediation pathway for the previously stated issues.²⁵ Scheme 2 portrays the liberation of CO₂ from the reaction of CBDA-AP-I and ethylene carbonate which drove high conversions to the hydroxyethyl substituted compound, denoted CBDA-AP-I-HE. The installed hydroxy ethyl functionality subsequently increased solubility in polar aprotic solvents enabling the synthesis of a new cyclobutane-containing PU, outlined in Scheme 3.

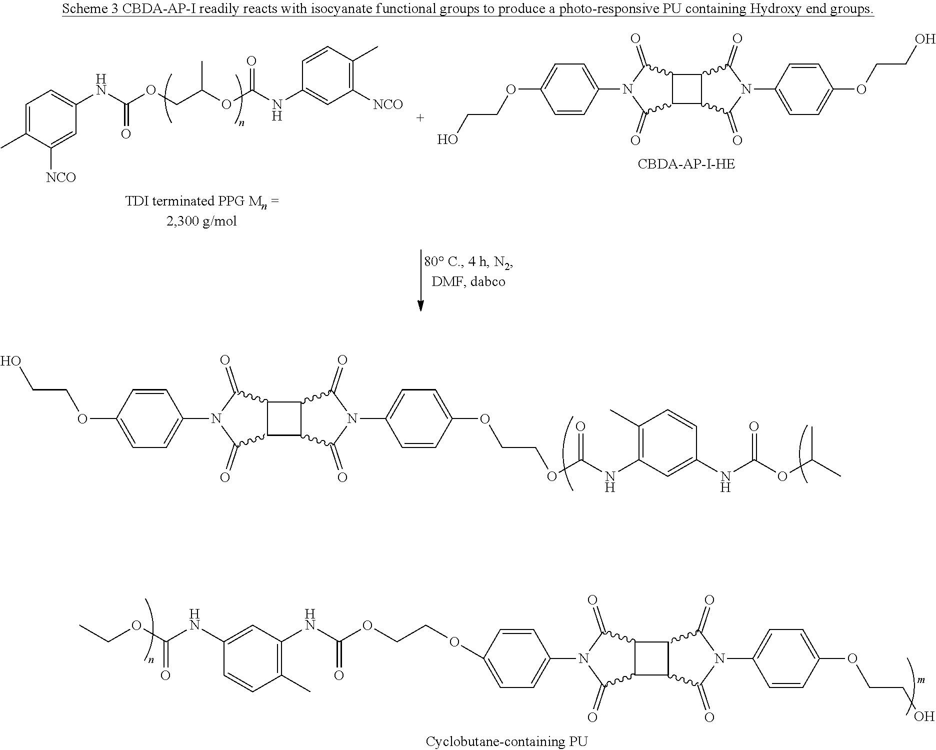

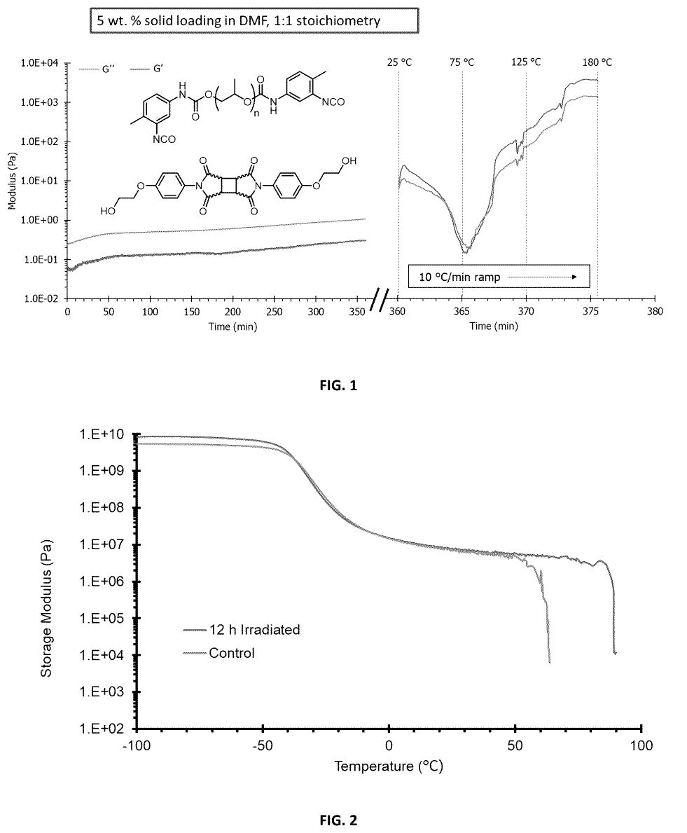

Common processing techniques for semiconductor TBDB applications include spin coating of the bonding compound followed by a thermal bake to complete the bonding process. A multi-step rheological experiment probed the efficacy of a CBDA-AP-I-HE polyurethane system for this application, exploring both the spin coating process and thermally-initiated polymerization process. A DMF solution containing both CBDA-AP-I-HE and PPG-TDI monomers underwent a steady shear experiment for 6 h to probe resin storage efficacy. Minimal viscosity increase over the 6 h period displayed the temporal stability of this monomer system. Subsequent heating mimicked a thermal bake process and demonstrated change in the storage modulus as temperature increased. A thermally induced viscosity decrease explains the initial dip in moduli values, however a modulus increase was observed as the temperature approached sufficient values to promote molecular weight gain. Continued molecular weight increase drove a subsequent increase in moduli values. A notable crossover between storage and loss moduli signaled the formation of a polymeric system that has predominantly solid, or elastic, characteristics. A relevant bake temperature of 180° C. produced a plateau for both the storage and loss moduli, which signaled a complete bake procedure and formation of the relevant polyurethane, shown in FIG. 1.

Rheological and spectroscopy data confirmed the synthesis of a CBDA-AP-I-HE PU containing a PPG-TDI soft segment, however further thermomechanical and spectroscopic investigations allowed for evaluating cyclobutane ring cleavage after irradiation with 254 nm light. DMA measured the thermomechanical behavior of the irradiated and control PU film, shown in FIG. 2. Interestingly, the DMA trace revealed the presence of a rubbery plateau, indicative of phase separation between the PPG soft segment (SS) and CBDA-AP-I-HE hard segment (HS). PU formulations often rely on chain extenders to build the molecular weight of the hard segment, resulting in the subsequent nano-scale phase separation, however the absence of chain extender in this PU formulation rendered this result surprising.^{26, 27} Both the control and irradiated PU displayed a tan δ peak at −27° C., corresponding to the PPG soft segment. This indicated that irradiation did not affect PPG interactions contained in the SS. However, irradiation resulted in an increase in flow temperature from 62° C. to 89° C. HS interactions, mainly hydrogen bonding, determine the ultimate transition temperature for an amorphous PU.^{28, 29} Thus, the change in flow temperatures between the irradiated and control samples indicates a change in the chemical nature or morphology of the cyclobutane-containing HS. Without wishing to be bound by theory, cleaving of the cyclobutane bisimide linkage and subsequent maleimide formation and polymerization explains the increase in flow temperature.¹³

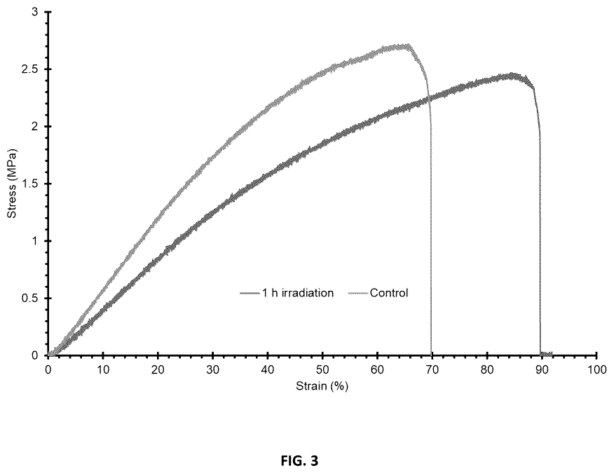

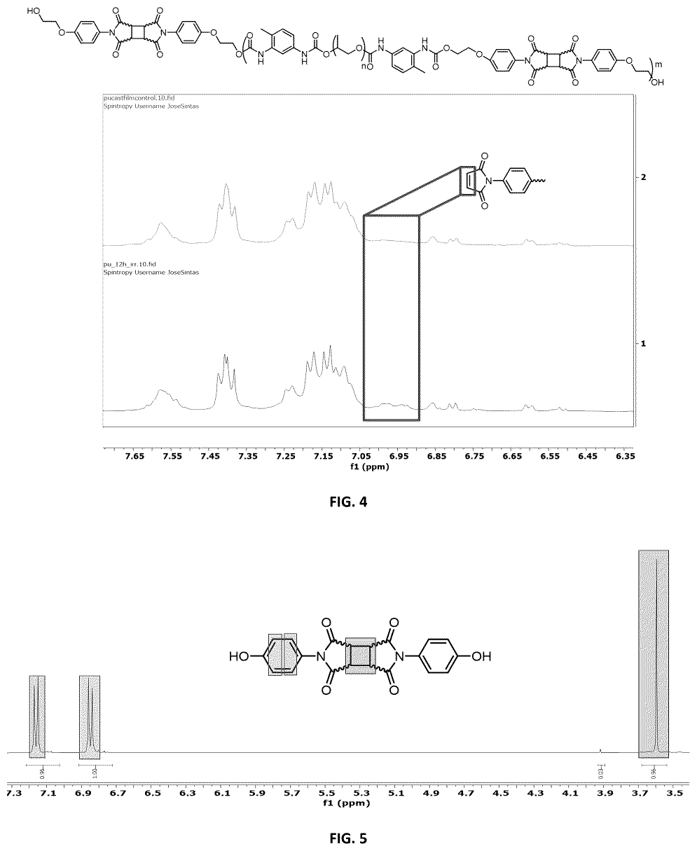

Further probing of mechanical properties revealed a change in the tensile curves for irradiated and non-irradiated PU samples, shown in FIG. 3. Stress-strain profiles for the control and irradiated sample display elastomeric properties consistent with DMA rubbery plateau moduli values. The elastomeric properties exhibited by CBDA-AP-I-HE PU provide consistent performance throughout a wide temperature range (80° C.). Without wishing to be bound by theory, chain extension from maleimide polymerization resulted in an increase of the strain at break after irradiation. It should be noted however, that the Young's modulus of the control film is larger due to higher molecular weight at a similar 25° C. storage modulus derived from DMA.

While thermomechanical measurements provided insight into the feasibility and effects of cyclobutane bisimide photo-cleavage, further probing with ¹H NMR spectroscopy enabled additional verification of this phenomenon. The appearance of maleimide protons downfield at ˜7 ppm indicated the successful ring opening of the cyclobutane bisimide moiety to produce alkene proton peaks. This appearance, in agrecance with thermomechanical data indicated partial cleavage of the cyclobutane ring. Low signal intensity indicated that only partial cleavage was obtained, however longer irradiation times can serve to increase the yield of this reaction. Likewise, conservation of the TDI peaks adjacent to the PPG groups revealed conservation of urethane linkages and an absence of PU degradation outside of the cyclobutane linkage.

### CONCLUSION

Selective addition of 4-aminophenol to CBDA enabled the synthesis of a new cyclobutane bisamic acid that underwent subsequent imidization to form a cyclobutane bisimide monomer containing bisphenol reactivity. Subsequent functionalization with ethylene carbonate yielded hydroxy ethyl functionality that allowed for the synthesis of thermally stable PUs containing cyclobutane bisimide linkages. The synthesized CBDA-AP-I-HE PU contained a PPG SS resulting in phase separation and a rubbery plateau with a storage modulus of 107 Pa and elastomeric properties confirmed with tensile data. Subsequent irradiation with 254 nm light resulted in a change in the HS chemistry derived from cyclobutane ring cleavage, resulting in an elevation of flow temperature and strain at break. Additionally, ¹H NMR revealed the appearance of maleimide protons after 254 nm irradiation further suggesting cyclobutane ring cleavage. Further studies that leverage a higher intensity of 254 nm irradiation can serve to increase the yield of cyclobutane cleavage and shorten irradiation times.

### REFERENCES

1. Bai, Y.; Li, K.; Huang, S.; Li, J.; Liu, Q.; Li, Z.; Wang, X.; Zhang, G.; Sun, R. In A Novel Process to Reduce the Warpage of Bond Pair for Temporary Bonding and De-bonding Technique, 2022 23rd International Conference on Electronic Packaging Technology (ICEPT), 10-13 Aug. 2022; 2022; pp 1-4.
2. Xu, D.; Wang, H. W.; Patel, J.; Brun, X. F.; Hirota, K.; Capsuto, E.; Kato, H.; Sugo, M. In A Novel Design of Temporary Bond Debond Adhesive Technology for Wafer-Level Assembly, 2020 IEEE 70th Electronic Components and Technology Conference (ECTC), 3-30 Jun. 2020; 2020; pp 68-74.
3. Haq, J.; Vogt, B. D.; Howard, E.; Loy, D., Temporary bond-debond technology for high-performance transistors on flexible substrates. Journal of the Society for Information Display 2010, 18 (11), 884-891.
4. Mo, Z.; Wang, F.; Li, J.; Liu, Q.; Zhang, G.; Li, W.; Yang, C.; Sun, R., Temporary Bonding and Debonding in Advanced Packaging: Recent Progress and Applications. Electronics 2023, 12 (7), 1666.
5. Huang, D.; Liu, J.; Li, J.; Wang, F.; Li, K.; Liu, Q.; Yin, H.; Zhang, G.; Sun, R., Controlled Thermal Imidization of Thermoplastic Polyimide for Temporary Bonding and Debonding in Advanced Packages. ACS Applied Polymer Materials 2022, 4 (11), 8508-8519.
6. Hermanowski, J. In Thin wafer handling-Study of temporary wafer bonding materials and processes, 2009 IEEE International Conference on 3D System Integration, 28-30 Sep. 2009; 2009; pp 1-5.
7. Hashiguchi, H.; Fukushima, T.; Murugesan, M.; Kino, H.; Tanaka, T.; Koyanagi, M., High-Thermoresistant Temporary Bonding Technology for Multichip-to-Wafer 3-D Integration With Via-Last TSVs. IEEE Transactions on Components, Packaging and Manufacturing Technology 2019, 9 (1), 181-188.
8. Lec, S.-W.; Lec, T.-H.; Park, J.-W.; Park, C.-H.; Kim, H.-J.; Kim, S.-M.; Lec, S.-H.; Song, J.-Y.; Lee, J.-H., The effect of laser irradiation on peel strength of temporary adhesives for wafer bonding. International Journal of Adhesion and Adhesives 2015, 57, 9-12.
9. June, S. M.; Suga, T.; Heath, W. H.; Long, T. E.; Lin, Q.; Puligadda, R., Photo-Reactive Polyimides and Poly(siloxane imide) s as Reversible Polymeric Interfaces. The Journal of Adhesion 2010, 86 (10), 1012-1028.
10. Moore, J. A.; Dasheff, A. N., An intrinsically photosensitive polyimide. Chemistry of Materials 1989, 1 (1), 163-166.
11. Hergenrother, P., The Use, design, synthesis, and properties of high performance/high temperature polymers: An overview, high perform. High Performance Polymers 2003, 15, 3-45.
12. Moyer, E. S.; Mohanty, D. K.; Shaw, J.; McGrath, J. E. In Synthesis and characterization of soluble photoimageable polyimide and poly(imide siloxane) homo- and copolymers, International SAMPE Symposium and Exhibition (Proceedings), 1989; pp 894-904.
13. Decker, C.; Bianchi, C.; Jönsson, S., Light-induced crosslinking polymerization of a novel N-substituted bis-maleimide monomer. Polymer 2004, 45 (17), 5803-5811.
14. Kang, M.; Moon, B., Synthesis of Photocleavable Poly(styrene-block-ethylene oxide) and Its Self-Assembly into Nanoporous Thin Films. Macromolecules 2009, 42 (1), 455-458.
15. Piggott, A. M.; Karuso, P., Synthesis of a new hydrophilic o-nitrobenzyl photocleavable linker suitable for use in chemical proteomics. Tetrahedron Letters 2005, 46 (47), 8241-8244.
16. Suzuki, H.; Abe, T.; Takaishi, K.; Narita, M.; Hamada, F., The synthesis and X-ray structure of 1,2,3,4-cyclobutane tetracarboxylic dianhydride and the preparation of a new type of polyimide showing excellent transparency and heat resistance. Journal of Polymer Science Part A: Polymer Chemistry 2000, 38 (1), 108-116.
17. Trenor, S. R.; Shultz, A. R.; Love, B. J.; Long, T. E., Coumarins in Polymers: From Light Harvesting to Photo-Cross-Linkable Tissue Scaffolds. Chemical Reviews 2004, 104 (6), 3059-3078.
18. Trenor, S. R.; Long, T. E.; Love, B. J., Photoreversible Chain Extension of Poly(ethylene glycol). Macromolecular Chemistry and Physics 2004, 205 (6), 715-723.
19. Hughes, T.; Simon, G. P.; Saito, K., Chemistries and capabilities of photo-formable and photoreversible crosslinked polymer networks. Materials Horizons 2019, 6 (9), 1762-1773.
20. Lentin, I.; Gorbunov, A.; Bezzubov, S.; Nosova, V.; Cheshkov, D.; Kovalev, V.; Vatsouro, I., Shrinkable/stretchable bis(calix [4] arenes) comprising photoreactive azobenzene or stilbene linkers. Organic Chemistry Frontiers 2023, 10 (6), 1470-1484.
21. Ichimura, K.; Akita, Y.; Akiyama, H.; Kudo, K.; Hayashi, Y., Photoreactivity of Polymers with Regioisomeric Cinnamate Side Chains and Their Ability To Regulate Liquid Crystal Alignment. Macromolecules 1997, 30 (4), 903-911.
22. Kaur, G.; Johnston, P.; Saito, K., Photo-reversible dimerisation reactions and their applications in polymeric systems. Polymer Chemistry 2014, 5 (7), 2171-2186.
23. Dennis, J. M.; Fahs, G. B.; Moore, R. B.; Turner, S. R.; Long, T. E., Synthesis and Characterization of Polysulfone-Containing Poly(butylene terephthalate) Segmented Block Copolymers. Macromolecules 2014, 47 (23), 8171-8177.
24. Sardon, H.; Pascual, A.; Mecerreyes, D.; Taton, D.; Cramail, H.; Hedrick, J. L., Synthesis of Polyurethanes Using Organocatalysis: A Perspective. Macromolecules 2015, 48 (10), 3153-3165.
25. Weyhrich, C.; Will, J.; Heifferon, K.; Brown, J.; Arrington, C.; Meenakshisundaram, V.; Williams, C.; Long, T., 3D-Printing of Poly(arylene ether sulfone) s: Functional High-Performance Polymers for Vat Photopolymerization. Macromolecular Chemistry and Physics 2022, 224.
26. Aneja, A.; Wilkes, G. L., A systematic series of ‘model’ PTMO based segmented polyurethanes reinvestigated using atomic force microscopy. Polymer 2003, 44 (23), 7221-7228.
27. Yilgör, I.; Yilgör, E.; Wilkes, G. L., Critical parameters in designing segmented polyurethanes and their effect on morphology and properties: A comprehensive review. Polymer 2015, 58, A1-A36.
28. Beniah, G.; Fortman, D. J.; Heath, W. H.; Dichtel, W. R.; Torkelson, J. M., Non-Isocyanate Polyurethane Thermoplastic Elastomer: Amide-Based Chain Extender Yields Enhanced Nanophase Separation and Properties in Polyhydroxyurethane. Macromolecules 2017, 50 (11), 4425-4434.
29. Cheng, B.-X.; Gao, W.-C.; Ren, X.-M.; Ouyang, X.-Y.; Zhao, Y.; Zhao, H.; Wu, W.; Huang, C.-X.; Liu, Y.; Liu, X.-Y.; Li, H.-N.; Li, R. K. Y., A review of microphase separation of polyurethane: Characterization and applications. Polymer T_{g}sting 2022, 107, 107489.

### Example 2

### Photo-Responsive Cyclobutene Bisimide-Containing Polysulfones Capable of Light-Mediated Depolymerization

### INTRODUCTION

Polysulfones (PSUs) represent a class of high-performance engineering polymers renowned for their desirable mechanical properties and high chemical resistance, biocompatibility, and steralizability.¹ These properties encouraged the use of PSUs in various membrane applications where anti-fouling and sterilization requirements exist.²⁻⁷ Additionally, their high strength-to-weight ratio encourages their use in automotive, aerospace, and electronic applications.^{8, 9} The aromatic structure of PSUs enables these desired engineering properties and is derived from a nucleophilic aromatic substitution reaction between bisphenol-A (BPA) and dichlorodiphenyl sulfone (DCDPS). Bulky substituents of the comprising monomers prevent crystallization, while restricting rotation along the backbone resulting in excellent optical clarity and high T_{g}.¹⁰ These excellent properties and wide spread use is however limited to traditional melt processing techniques and thus possesses room for innovation.

While PSUs display inherent instability to UV irradiation, installing a governable light-mediated degradation pathway remains desired for temporal control over molecular weight.¹¹ Control over these processes impacts both semi-conductor patterning and encapsulant applications, both of which stand to benefit from the thermomechanical performance and chemical resistance of PSUs. Recent literature describes the use of PSUs as encapsulants for metathesis catalysts.¹² They displayed good retention and release of catalyst, below and above the T_{g} respectively, through temperature modulation. The released catalyst subsequently initiated a depolymerization process of polybutadiene rubbers that resulted in a stable trigger for tire depolymerization that is easily imbedded into the final product. Likewise, additive manufacturing benefits from controlled catalyst release. Namely, recent advancements in frontal polymerization involved the use of a PSU encapsulant for greater resin storage life, preventing spontancous polymerization.¹³ While these innovations provide exciting avenues for future PSU research, a thermal release trigger limits these applications to high energies and poor spatial control. Thus, a photo-mediated alternative remains desired for these systems as it provides both spatial and temporal control over release in addition to orthogonality to thermal stimuli.¹⁴

Semi-conductor packaging applications leverage photo-mediated processing techniques on an industrial scale.¹⁵⁻¹⁷ Structural adhesives for bond/debond applications as well as positive photoresist for patterning benefit from photo-degradation processes.¹⁸⁻¹⁹ The ability to cleave covalent bonds with light provides on-demand molecular weight control, which subsequently modifies mechanical properties and solubility. In addition to positive photoresist applications, photo-degradability plays an important role in next generation semiconductor processing applications including the bond/debond process where a silicon wafer is bound to a glass substrate. The release of the silicon wafer after patterning is often mediated with heat, which results in chip warpage or delamination. Thus, a photonic trigger that circumvents thermal warpage is desired.

Various [2+2] cycloaddition/cleavage modalities exist for photo-mediated molecular weight control including coumarin, thymine, cinnamic, and stilbene chemistries.²⁰ While these chemistries possess efficient photo-reversible functions, thermal stability and short, high-energy irradiation windows are required to ensure orthogonality to other processes. Previous research demonstrated the feasibility of Cyclobutane bisimide photo-reversable chemistry for photo-cleaving semiconductor applications.^{21, 22} Outstanding thermal properties of imide and PSU chemistries allow for facile transfer of this chemistry to catalyst encapsulation applications. Thus, the facile synthesis of a cyclobutane bisimide monomer that contains bisphenol reactivity enabled the installation of photo-cleavable functionality along the PSU backbone that retains thermal stability. These new PSUs retained thermomechanical properties expected from conventional BPA and DCDPS copolymers. Additionally, ¹H NMR analysis revealed cyclobutane bisimide end groups resulting from the decreased reactivity of the cyclobutane bisimide monomer and unfavored addition compared to BPA. This phenomenon added potential for segmented PSU block copolymers capable of selective block cleavage.²³ Lastly, irradiation with 254 nm light resulted in a molecular weight decrease that correlated with cyclobutane incorporation.

### Materials and Methods

### Materials

Cyclobutane-1,2,3,4-tetracarboxylic dianhydride (CBDA) was graciously provided by Jayhawk Fine ChemicalsX and used as received. 4-Aminophenol (>98%), deuterated dimthylsulfoxide-d6 (99.95%), deuterated chloroform CDCl₃ (99.9%), potassium carbonate (K₂CO₃) (>99%), bisphenol A (>99%), 4,4′-dichlorodiphenyl sulfone (DCDPS) (>98%), N,N-dimethylacetamide (DMAc) (anhydrous, 99.8%), and Celite×545 filter agent, were purchased from SigmaAldrich and used as received. Hydrochloric acid, chloroform (HPLC grade), 1,2-dichlorobenzene (>99%), toluene, methanol, and tetrahydrofuran (THF) were purchased from Fisher Chemical and used as received.

### Instruments

¹H and ¹³C nuclear magnetic resonance (NMR) spectroscopy utilized a Bruker Avance NEO 500 MHz spectrometer functioning at 500.15 MHz and 23° C. (solution concentration of 10 mg mL 1). High temperature NMR was run on a Varian MR400 at 80° C. for the CBDA-AP-I monomer. A ThermoFisher Scientific Nicolet iS10 FTIR spectrometer, with a diamond cell at 25° C., identified key stretches for synthesized monomers and polymers. A TA Instruments TGA 5500 facilitated thermogravimetric analysis (TGA) by utilizing a heating rate of 10° C. min 1 from 25 to 600° C. with a steady nitrogen purge. The T_{d.5}%, or temperature where 5% of the original sample mass was lost, served as an indicator for sample thermal stability. A TA Instruments DSC 2500 with heat/cool/heat cycles of 10° C. min 1 provided differential scanning calorimetry data where the sample was under a nitrogen environment throughout the experiment. DSC provided the glass transition temperatures (TgS) from the midpoint of the endothermic transition in the second heat. A TA Instruments DMA Q800 with a temperature ramp of 3° C. min 1 from −90° C. to 180° C. at 1 Hz provided a T_{g} taken from the peak in the tan delta. Liquid nitrogen cooling permitted the cryogenic temperatures required to observe the thermal transitions of the PSU films. A Mineralight 254 nm 15 w lamp equipped with a 254 nm filter enabled consistent irradiation of samples at an intensity of 4.8 mW/cm². A Waters ARC HPLC size exclusion chromatography system operating at a flow rate of 1 mL/min at 5 mg/mL and temperature of 50° C. enabled molecular weight determination for polymeric samples. Samples were run in 0.05 M LiBr DMF and passed through a 0.45 micrometer filter before injecting. A Waters dRI detector monitored concentration change and final molecular weight was determined against a polystyrene calibration curve.

### Synthesis of CBDA-AP-I

CBDA (10.00 g, 51.0 mmol), 4-aminophenol (11.13 g, 102.0 mmol), 100 mL of toluene and 500 mL of 1,2-dichlorobenzene were added to a 3-neck round bottomed flask equipped with a magnetic stir bar, nitrogen inlet, reflux condenser, and Dean-Stark apparatus. Reagents were allowed to mix and subsequently heated to 180° C. The reaction was allowed to progress for 18 h and remained heterogenous throughout. The toluene served to remove any water produced throughout the imidization reaction through an azeotrope and the Dean-Stark apparatus was emptied as needed. The precipitate was filtered using a fritted funnel and allowed to dry overnight then recrystallized in DMF. The final isolated yield was >90%. ¹H NMR (DMSO-d6, 80° C., δ, FIG. 15) 9.77 (s, 2H), 7.2 (d, 4H, J=8.5 Hz), 6.87 (d, 4H, J=8.5 Hz), 3.62 (s, 4H).

### Synthesis of Phenol-terminated PSU 1, 2, and 3

Phenol-terminated polysulfones (PSUs) were synthesized using an established procedure, illustrated in Scheme 4.^{11. 24} In order to achieve phenolic ends (preferred over DCDPS end groups for stability), a slight excess of phenol monomer was employed. PSU 1 is a homopolymer of BPA and DCDPS and thus only utilized those two monomers. However, PSU 2 and 3 contained either 5 mol. % or 10 mol. % respectively of CBDA-AP-I relative to the total moles of bisphenol incorporated. BPA, or a combination of BPA and CBDA-AP-I at either a 95:5 or 90:10 molar ratio respectively (0.2300 mol), 4,4′-dichlorodiphenyl sulfone (64.89 g, 0.2260 mol), and potassium carbonate (40.35, 0.2919 mol) were added to anhydrous N,N-dimethylacetamide (400 mL) and toluene (200 mL) in a three-necked, round-bottomed flask fitted with a nitrogen adapter, Dean-Stark trap with a condenser, and a glass mechanical stir rod with a Teflon® paddle. The heterogenous solution was purged with nitrogen for 20 min then heated to 160° C. The reaction was refluxed for 5 h, the toluene/water azeotrope was removed periodically, then the reaction was heated to 180° C. for 18 h. The resulting solution was filtered through a CeliteR earth plug, neutralized with 1 M HCl solution in THF, and then precipitated into methanol. The resulting white powder was dried in vacuo at 190° C. for 18 h. For PSU 1, 1H NMR spectroscopy elucidated molecular weight through end group analysis. ¹H NMR (500 MHZ, CDCl₃) ò 7.86-7.83 (d, 56H, J-8.89 Hz), 7.25-7.23 (d, 56H, J=8.75 Hz), 7.10-7.07 (d, 4H, J=8.67 Hz), 7.01-6.99 (d, 56H, J=8.87 Hz), 6.95-6.92 (d, 56H, J=8.71 Hz), 6.77-6.74 (d, 4H, J=8.71 Hz), 1.69 (s, 76H), 1.65 (s, 12H).

### Results and Discussion

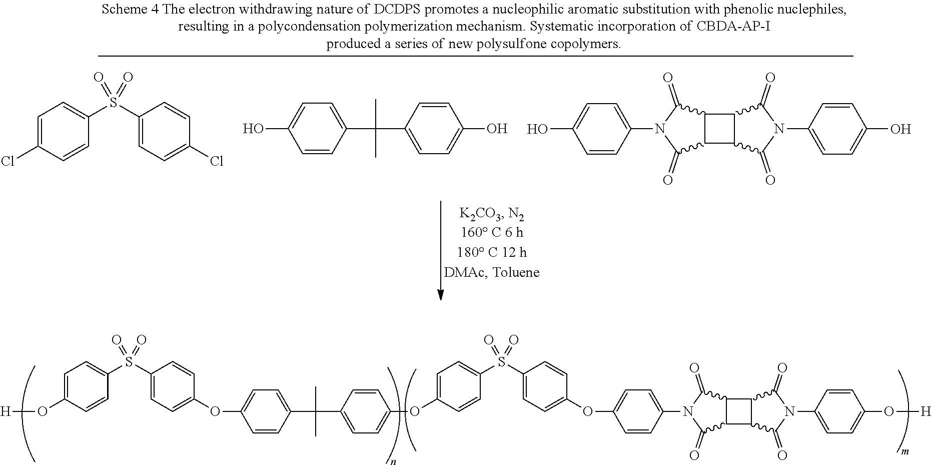

The favorable solubility of CBDA-AP-I in DMAc allowed for the retention of reaction conditions used to synthesize PSU 1. This proved fortuitous, as it eliminated deviations in reaction setups between all PSUs with consistent reaction setups. The phenolic reactivity of CBDA-AP-I enabled seamless incorporation of a cyclobutane bisimide into PSUs alongside BPA, a common PSU monomer. Furthermore, incorporation of varying concentrations of CBDA-AP-I, 5 mol. % for PSU 2 and 10 mol. % for PSU 3, allowed for systematic probing of structure-property relationships that arise due to the incorporation of this new monomer. A DMAc-toluene solvent selection provided both effective removal of water throughout the reaction facilitated by a toluene-water azeotrope as well as complete solubility of the growing polymer. A non-nucleophilic base enabled efficient trapping of the HCl condensate, driving the reaction towards high conversion. A silica plug and methanol precipitation removed impurities and provided high molecular weight PSUs capable of producing creasible films after melt-processing.

End group analysis is a well-established method for determining molecular weight of polymers.²⁵ Commonly, the proton integration of a known polymer terminal functionality is compared to known backbone proton integration, providing a degree of polymerization that is then converted to molecular weight. For PSU 1, which does not contain CBDA-AP-I, this method was effective at determining the molecular weight and agreed favorably with SEC analysis. PSU 2 and 3, however, did not display any discernable end group protons from either the BPA or CBDA-AP-I end groups. This is surprising, as an excess of phenol was leveraged to target phenolic end groups. Likewise, the high molecular weight of the resulting polymer suggests large degrees of conversion that would allow for these end groups to exist. Thus, without wishing to be bound by theory, not only are the end groups of PSU 2 and 3 CBDA-AP-I, but also that those end group protons overlap with backbone protons resulting in a peak convolution that prohibits molecular weight determination with NMR. This is further bolstered by the comparatively lower reactivity of CBDA-AP-I compared to BPA. Namely, BPA does not posses any electron withdrawing character that affects the reactive phenol moiety, while CBDA-AP-I possess an electron withdrawing imide linkage that decreases reactivity. For these reasons, it is likely that PSU 2 and 3 are gradient block copolymer, with the less reactive CBDA-AP-I monomers located in increasing concentration closer to the end groups of the polymer.

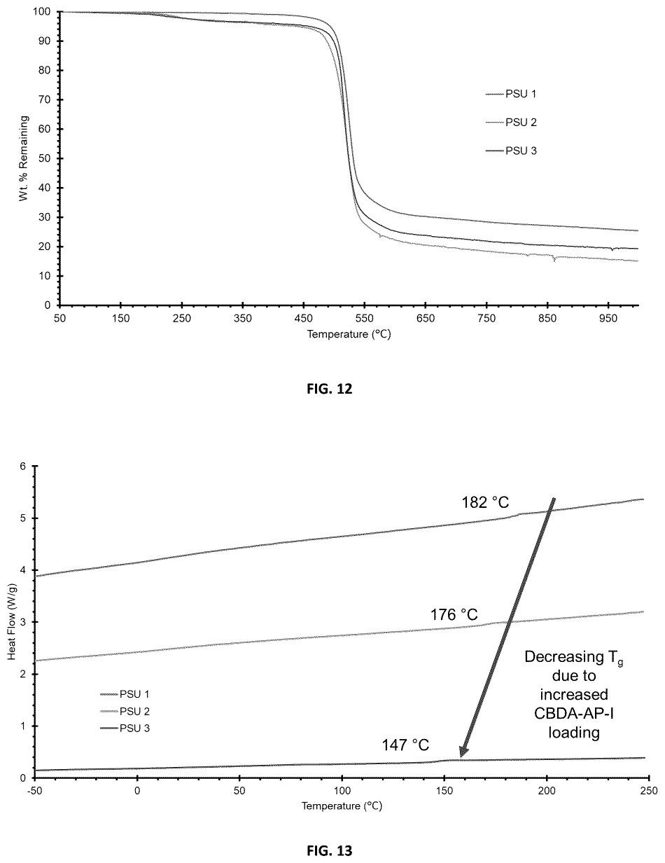

While NMR is effective at determining chemical structure, additional spectroscopic methods serve to bolster molecular structure analysis. FTIR enabled further probing of CBDA-AP-I incorporation. The distinct bisimide chemical group introduces a new C═O stretching mode that is distinct from the sulfone carbonyl system. PSU 1 did not display any imide symmetric or asymmetric carbonyl stretching modes, however PSU 2 and 3 possessed a carbonyl peak in the expected region. Furthermore, the stretch intensity increased from PSU 2 to PSU 3 as a result of the greater CBDA-AP-I incorporation. This data, coupled with NMR analysis, indicates that not only was CBDA-AP-I systematically incorporated into the PSU backbone, but is also less reactive than its BPA counterpart thus directing its position near the PSU end groups. This claim is further supported by thermogravimetric analysis (TGA). The stability of the polymer backbone, largely due to its restricted mobility, results in the majority of polymer thermal degradation to originate in the more mobile chain ends. TGA analysis of the CBDA-AP-I monomer demonstrates a thermal degradation temperature of 400° C., shown in FIG. 12. This can be counter intuitive, however the cyclobutane bisimide moiety is thermally robust. A systematic study on the weight loss profiles for all PSUs allowed further exploration of the effects CBDA-AP-I concentration had on the final polymer. PSU 2 and 3 displayed an earlier onset of weight loss, perhaps due to the electron withdrawing character of the bisimide linkage resulting in a weaker backbone linkage and premature degradation of the PSU backbone. It should be noted that the low quantity of weight loss does not directly correlate to wt. % CBDA-AP-I incorporation, however. Overall, the change in weight loss profile, loss of BPA chain ends on NMR, and appearance of imide stretching absorbances on FTIR strongly suggests controlled incorporation of CBDA-AP-I into the PSU backbone.

Differential scanning calorimetry (DSC) provided insight into the thermal properties CBDA-AP-I endows PSUs, shown in FIG. 13. PSU 1 possess a T_{g} at an expected temperature range (182° C.) when compared to literature values. Interestingly, however, the incorporation of rigid CBDA-AP-I did not raise T_{g} as expected, but lowered it considerably. This phenomenon trends with the concentration of CBDA-AP-I incorporated with PSU 2 having a higher T_{g} than PSU 3. While counterintuitive, polymer physics help explain this behavior. PSUs are commonly viewed as rigid rod, amorphous polymers and derive high T_{g}s from intermolecular entanglements that come from their helical structure.²⁶⁻²⁷ These well-defined helical structures produce a very short entanglement molecular weight, however require a regular repeating chemical structure to produce this property. The addition of CBDA-AP-I in PSU 2 and 3 disrupt this helical structure, thus decreasing the beneficial entanglement interactions PSU 1 enjoys. As previously discussed, the CBDA-AP-I monomer is located near the end of the PSU chains. This directly affects T_{g} because chain mobility is theorized to start at the less sterically hindered chain ends.

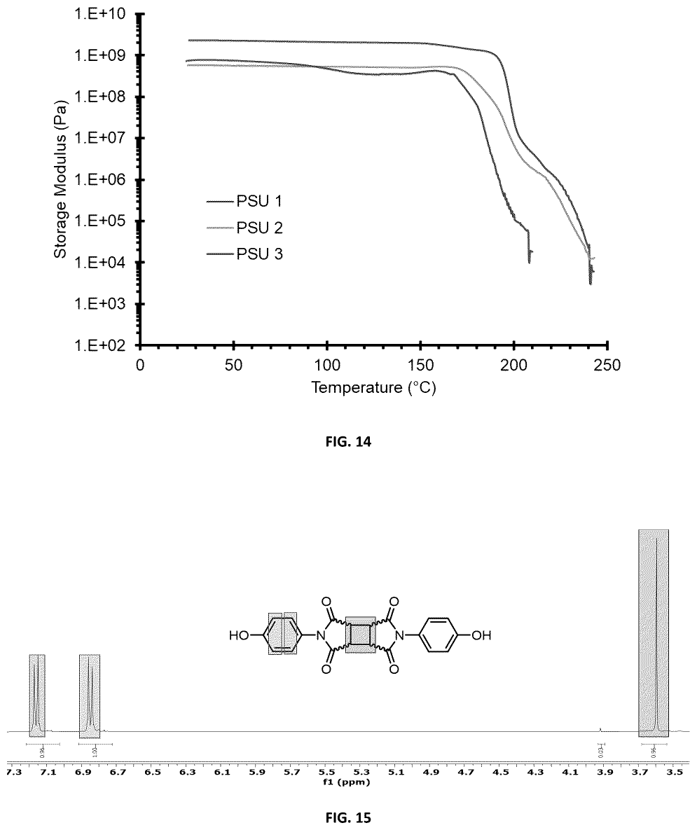

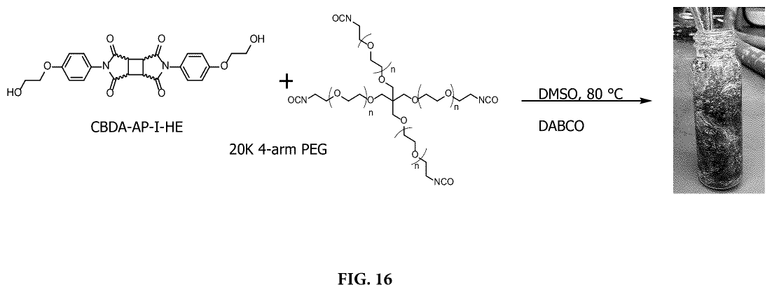

Dynamic mechanical analysis allows for deeper exploration into the effects CBDA-AP-I concentration has on thermomechanical properties, displayed in FIG. 14. PSU 1 displayed a possess an expected thermomechanical behavior of an amorphous, high T_{g} polymer with a tan δ peak at 200° C. PSU 1 also displays a storage modulus value >1 Gpa, characteristic of a glassy polymer. PSU 2 and 3, however, displayed lower modulus values suggesting a low temperature transition not captured in this DMA experiment. Likewise, the decrease in T_{g} observed in DSC experiments is reflected in the DMA traces with tan & peaks for PSU 2 and 3 T_{g}s at 198 and 188° C. respectively. Additionally, PSU 3 which contains a 10 mol. % concentration of CBDA-AP-I displays an additional tan δ peak at 111° C. DSC analysis does not display this transition; however, a faster scanning rate can help resolve this. This DMA curve supports previous claims that the incorporation of a rigid cyclobutane bisimide comonomer can serve to decrease chain entanglements, thus lowering thermal properties. Furthermore, the possibility of a gradient copolymer topology with CBDA-AP-I located near the chain ends can result in some degree of phase separation, leading to two independent T_{g}s at higher concentrations of the cyclobutane monomer.

Lastly, these PSUs were evaluated for their ability to photocleave under 254 nm irradiation. The incorporated cyclobutane bisimide moiety endows photo-reactivity when exposed to certain wavelengths, namely the photocleavage of the cyclobutane ring under 254 nm irradiation to form 2 maleimide end groups. A 254 nm filter attached to a broadband UV light source and size exclusion chromatography (SEC) provided an evaluation of PSU molecular weight before and after irradiation for all samples. Even in the absence of cyclobutane bisimide monomers, PSUs react with UV light to undergo several chain scission events through the formation of radicals.²⁸ Another result of radical formation commonly experienced in PSUs is the coupling reaction of several radicals to provide chain extension after UV irradiation. These oxidation events lead to a decrease in material properties, namely toughness. UV irradiation of PSU 1 revealed a chain coupling degradation pathway, seen by the increase in molecular weight after narrow 254 nm irradiation of a melt pressed thin film, Table 1. Interestingly, the addition of CBDA-AP-I in PSU 2 and 3 resulted in a noticeable decrease in Mₙ after 254 nm irradiation. This is due to the incorporation of the cyclobutane bisimide functional group along the polymer backbone that underwent photocleavage. As expected, the change in molecular weight increased with increasing CBDA-AP-I loading (27% for PSU 2 and 34% for PSI 3).

TABLE 1
SEC reveals change in molecular weight after irradiation
with 254 nm light. The decrease in molecular weight in
PSU 1 and 2 is attributed to the introduction of controlled
photo-cleaving of cyclobutane bisimide linkages.

| Polymer | Mn (g/mol) | Mx (g/mol) | Ð |
|---|---|---|---|
| PSU 1 (0 mol. %) | 14,600 | 27,600 | 1.88 |
| PSU 2 (5 mol. %) | 31,500 | 57,200 | 1.63 |
| PSU 3 (10 mol. %) | 24,800 | 31,000 | 1.25 |
| PSU 1 L (0 mol. %) | 19,800 | 33,100 | 1.68 |
| PSU 2 L (5 mol. %) | 23,000 | 20,700 | 1.77 |
| PSU 3 L (10 mol. %) | 16,300 | 22,100 | 1.36 |

| Polymer | ΔMn (g/mol) | ΔMn (%) | Δ Ð |
|---|---|---|---|
| PSU 1 (0 mol. %) | +5,200 | +36% | −0.20 |
| PSU 2 (5 mol. %) | −8,500 | −27% | +0.14 |
| PSU 3 (10 mol. %) | −8,500 | −34% | +0.11 |

### Conclusions:

A polycondensation synthetic strategy afforded a series of new PSUs containing photoreactive cyclobutane bisimide linkages. The incorporation of a new monomer, CBDA-AP-I, provided insight into new structure-property relationships for PSUs, evaluated with DSC, DMA, FTIR, and NMR analysis. Spectroscopy revealed the successful installation of CBDA-AP-I into the polymer backbone, with NMR indicating an increase in CBDA-AP-I concentration towards the end groups of the polymer. Electron withdrawing imide linkages in resonance with the reactive phenols of this compound can explain this phenomenon. This without wishing to be bound by theory, produced a gradient copolymer topology where CBDA-AP-I reacts after BPA yielding CBDA-AP-I end groups. Likewise, the incorporation of CBDA-AP-I resulted in a trend of decreasing T_{g} with increasing concentration. DMA also indicated the possibility of phase separation at an adequate concentration of the CBDA-AP-I monomer, as noted by the two tan δ peaks present in PSU 3. Further experiments that utilize X-ray scattering can explore this phenomenon, however. Lastly, irradiation with 254 nm light showed a clear decrease in molecular weight within PSU 2 and 3, indicating photocleavage of the incorporated cyclobutane bisimide linkage.

### REFERENCES

1. Deberdeev, T. R.; Akhmetshina, A. I.; Karimova, L. K.; Ignat′eva, E. K.; Galikhmanov, N. R.; Grishin, S. V.; Berlin, A. A.; Deberdeev, R. Y., Aromatic Polysulfones: Strategies of Synthesis, Properties, and Application. Polymer Science, Series D 2020, 13 (3), 320-328.
2. Serbanescu, O. S.; Voicu, S. I.; Thakur, V. K., Polysulfone functionalized membranes: Properties and challenges. Materials Today Chemistry 2020, 17, 100302.
3. Tan, X.; Rodrigue, D., A Review on Porous Polymeric Membrane Preparation. Part I: Production Techniques with Polysulfone and Poly(Vinylidene Fluoride). Polymers 2019, 11 (7), 1160.
4. Abdelrasoul, A.; Doan, H.; Lohi, A.; Cheng, C.-H., Morphology Control of Polysulfone Membranes in Filtration Processes: a Critical Review. ChemBioEng Reviews 2015, 2 (1), 22-43.
5. Kheirich, S.; Asghari, M.; Afsari, M., Application and modification of polysulfone membranes. Reviews in Chemical Engineering 2018, 34 (5), 657-693.
6. Chen, Y.; Lin, B.; Qiu, Y., Modification of polysulfone and the biomedical application of modified polysulfone. International Journal of Polymeric Materials and Polymeric Biomaterials 2023, 72 (3), 224-242.
7. Hoseinpour, V.; Noori, L.; Mahmoodpour, S.; Shariatinia, Z., A review on surface modification methods of poly(arylsulfone) membranes for biomedical applications. Journal of Biomaterials Science, Polymer Edition 2021, 32 (7), 906-965.
8. Yu, L.; Zhao, D.; Wang, W., Mechanical properties and long-term durability of recycled polysulfone plastic. Waste Management 2019, 84, 402-412.
9. Andrady, A. L.; Neal, M. A., Applications and societal benefits of plastics. Philos Trans R Soc Lond B Biol Sci 2009, 364 (1526), 1977-84.
10. Hergenrother, P., The Use, design, synthesis, and properties of high performance/high temperature polymers: An overview, high perform. High Performance Polymers 2003, 15, 3-45.
11. Weyhrich, C.; Will, J.; Heifferon, K.; Brown, J.; Arrington, C.; Meenakshisundaram, V.; Williams, C.; Long, T., 3D-Printing of Poly(arylene ether sulfone) s: Functional High-Performance Polymers for Vat Photopolymerization. Macromolecular Chemistry and Physics 2022, 224.
12. Warner, M. J.; Lassa, J. P.; Narcross, H.; Commisso, A.; Ghosh, K.; Romero, M.; Schwartz, J. M.; Engler, A. C.; Kohl, P. A.; Leguizamon, S. C.; Jones, B. H., Chemical Recycling of Polybutadiene Rubber with Tailored Depolymerization Enabled by Microencapsulated Metathesis Catalysts. ACS Sustainable Chemistry & Engineering 2023, 11 (39), 14538-14548.
13. Davydovich, O.; Greenlec, A. J.; Root, H. D.; Jansen, A. L.; Gallegos, S. C.; Warner, M. J.; Kent, M. S.; Cardenas, J. A.; Appelhans, L. N.; Roach, D. J.; Jones, B. H.; Leguizamon, S. C., Encapsulated Transition Metal Catalysts Enable Long-term Stability in Frontal Polymerization Resins. Macromolecules 2023, 56 (18), 7543-7550.
14. Yoon, T. P.; Ischay, M. A.; Du, J., Visible light photocatalysis as a greener approach to photochemical synthesis. Nature Chemistry 2010, 2 (7), 527-532.
15. Moreau, W. M., Semiconductor Lithography: Principles, Practices, and Materials. Springer US: 2012.
16. Costner, E. A.; Lin, M. W.; Jen, W.-L.; Willson, C. G., Nanoimprint Lithography Materials Development for Semiconductor Device Fabrication. Annual Review of Materials Research 2009, 39 (1), 155-180.
17. Lin, B.-J., Immersion lithography and its impact on semiconductor manufacturing. Journal of Micro/Nanolithography, MEMS, and MOEMS 2004, 3 (3).
18. Haq, J.; Vogt, B. D.; Howard, E.; Loy, D., Temporary bond—debond technology for high-performance transistors on flexible substrates. Journal of the Society for Information Display 2010, 18 (11), 884-891.
19. Ober, C. K.; Käfer, F.; Yuan, C., Recent developments in photoresists for extreme-ultraviolet lithography. Polymer 2023, 280, 126020.
20. Hughes, T.; Simon, G. P.; Saito, K., Chemistries and capabilities of photo-formable and photoreversible crosslinked polymer networks. Materials Horizons 2019, 6 (9), 1762-1773.
21. June, S. M.; Suga, T.; Heath, W. H.; Long, T. E.; Lin, Q.; Puligadda, R., Photo-Reactive Polyimides and Poly(siloxane imide) s as Reversible Polymeric Interfaces. The Journal of Adhesion 2010, 86 (10), 1012-1028.
22. Moore, J. A.; Dasheff, A. N., An intrinsically photosensitive polyimide. Chemistry of Materials 1989, 1 (1), 163-166.
23. Dennis, J. M.; Fahs, G. B.; Moore, R. B.; Turner, S. R.; Long, T. E., Synthesis and Characterization of Polysulfone-Containing Poly(butylene terephthalate) Segmented Block Copolymers. Macromolecules 2014, 47 (23), 8171-8177.
24. Dennis, J. M.; Fahs, G. B.; Moon, N. G.; Mondschein, R. J.; Moore, R. B.; Wilkes, G. L.; Long, T. E., Synthesis of Polysulfone-Containing Poly(butylene terephthalate) Segmented Block Copolymers: Influence of Segment Length on Thermomechanical Performance. Macromolecules 2017, 50 (13), 5107-5113.
25. Coleman, M. M., Fundamentals of Polymer Science: An Introductory Text, Second Edition. CRC Press: 2019.
26. Yang, H.; Wu, X.; Li, S., A compatibilizer for a blend of polysulfone with a liquid crystalline polyester. Polymer Engineering & Science 1996, 36 (22), 2781-2784.
27. Shaw, M. T.; Miller, J. C., The rheology of polysulfone. Polymer Engineering & Science 1978, 18 (5), 372-377.
28. Yamashita, T.; Tomitaka, H.; Kudo, T.; Horie, K.; Mita, I., Degradation of sulfur-containing aromatic polymers: Photodegradation of polyethersulfone and polysulfone. Polymer Degradation and Stability 1993, 39 (1), 47-54.

### Example 3

### Non-Limiting Aspects of the Disclosure

An aspect of the technology described herein is a new cyclobutane bisimide-based monomer that can be used to make photocleavable polymers. New materials capable of supporting technically challenging patterning processes continue to be innovated in the semiconductor industry. Materials capable of being debonded using non-thermal techniques, such as using photodegradation, is one critical area. The system developed herein uses a photocleavable cyclobutane bisimide moiety that is synthetically versatile to be able to be used to assemble a variety of different photodegradable polymers that can be tuned to specific applications.

A difunctional alcohol capable of imparting photocleavage into a polymeric system was synthesized for semiconductor packaging or reversible bonding applications. This monomer is comprised of a cyclobutane bisimide ring that undergoes photocleavage when irradiated with 254 nm light.

As competitive innovation continues in the semiconductor industry, new materials capable of supporting technically challenging patterning processes continue to be innovated. These materials must often times meet diverse thermomechanical and optical requirements needed for multi-step fabrication processes, with an emphasis on thermal expansion coefficients, thermal stability, off-gassing, and photoresponsiveness. One such use case is the temporary bonding of a substrate on a supporting layer during the fabrication process. This involves an initial bonding process between a glass substrate and the wafer that subsequently undergoes a packaging process. The wafer must then be released from the substrate through a debond process that involves a release mechanism and washing of the bonding material off the wafer. A facile release mechanism of the bond/debond process is needed to ensure minimal damage to the wafer surface. Thermal debonding of the adhesive often requires elevated temperatures and heat cycling that leads to warpage or cracking of the processed device. For this reason, a departure from traditional thermal process is desired.

Light-based debonding techniques have received considerable attention due to lower processing costs and higher control. Research has indicated cyclobutane bisimide functionalities for bond/debond and patterning applications. Among structural adhesives, the imide functionality displays advanced thermal stability along with low static permittivity, chemical stability, and dielectric properties that result in their prolific use in the semiconductor sector. There exists research into photo-degradable polymers for application to semiconductor patterning and processing. These systems prove useful in various steps of the semiconductor packaging process due to their photo-crosslinking or photo-degradation potential.

A technology that is readily applied to a wide variety of polymeric systems is desired. Thus, the synthesis of a cyclobutane bisimide-containing monomer capable of photo-degradation is desired. The greater reactivity of the nucleophilic aryl amine of 4-aminophenol enabled a selective addition to CBDA, producing a cyclobutane-containing amic acid with bisphenol functionality. Subsequent imidization resulted in the cyclobutane-bisimide moiety with bisphenol reactivity. Furthermore, reacting this imide with ethylene carbonate resulted in the installation of hydroxy ethyl groups allowing for the selection of phenol or hydroxyl reactivity. This expands the application space for this material, broadening the scope of this monomer to both fully aromatic and aliphatic systems. Further polymerization of this monomer with diisocyanates resulted in the synthesis of a new, cyclobutane bisimide-containing polyurethane.

### INTRODUCTION

This innovation pertains to light-assisted semiconductor processing at the wafer level. It is targeted towards bond/debond applications to replace thermal debond procedures with light mediated ones. Additionally, this technology has application potential as a positive photo resist.

### Non-Limiting Description

Non-limiting synthetic procedures comprise:

Cyclobutane dianhydride (10.00 g, 51.0 mmol), 4-aminophenol (11.13 g, 102.0 mmol), 100 mL of toluene and 500 mL of 1,2-dichlorobenzene were added to a 3-neck round bottomed flask equipped with a magnetic stir bar, nitrogen inlet, reflux condenser, and Dean-Stark apparatus. Reagents were allowed to mix and subsequently heated to 180° C. The reaction was allowed to progress for 18 h and remained heterogenous throughout. The toluene served to remove any water produced throughout the imidization reaction through an azeotrope and the Dean-Stark apparatus was emptied as needed. The precipitate was filtered using a fritted funnel and allowed to dry overnight then recrystallized in DMF.

Likewise, the amic acid precursor can be made first by reacting 4-aminophenol with cyclobutane dianhydride to produce the subsequent amic acid. Heating of this monomer to 300° C. then provides a fully imidized product.

For producing a diol as opposed to a bisphenol:

CBDA-AP-I (5 g, 13.21 mmol), ethylene carbonate (3.49 g, 39.64 mmol) and TEA (4.01 g, 39.64 mmol) were added to a 2-necked 1000 mL round bottomed flask equipped with a nitrogen inlet, magnetic stir bar, and reflux condenser. 500 mL of DMF was added and heated to 120° C. under nitrogen flow. The reaction was allowed to proceed for 24 h before precipitating into ethyl acetate. The precipitate was collected with a fritted funnel and dried under reduced pressure overnight at 120° C.

After synthesis, the polymer is spin coated onto a wafer, bonded and dried. After the wafer is processed, the substrate is irradiated with 254 nm light, resulting in decreased adhesion and release of the wafer module.

### Non-Limiting, Surprising Aspects of the Disclosure

While cyclobutane bisimides are known for their photo-degradation and have been incorporated into polymeric systems, the formation of a cyclobutane-containing monomer that possess phenol or hydroxyl functionalities is new. This invention now enables the incorporation of photo-degradation into a wider variety of polymers, no longer limiting it to the polyimide space.

### Non-Limiting Advantages

Temperature stability along with degradability allows for harsh processing conditions while maintaining facile workup. Likewise the developed monomer possesses a larger application scope, enabling incorporation into more polymeric systems that allows for a broader range of properties while maintaining photo-degradability.

### Example 4

High performance cyclobutene bisimide-containing polyesters

### Brief, Non-Limiting Summary

While PSUs, and to a greater extent PUs, encompass a large share of polymer production, the large global production of polyesters renders them an important member of the plastics family. Thus, we can adapt this versatile monomer to polyesters.

The polycondensation between CBDA-AP-I-HE and terephthalic acid will synthesize a new polyester containing a photo-sensitive trigger placed along the polymer backbone, described in Scheme 5. This is advantageous, as polyesters already possess sustainable aspects including facile depolymerization and repolymerization.¹³⁻¹⁵ Likewise, the rise of bio-derived polyester monomers further solidifies polyesters as a sustainable platform.¹⁴ The addition of a thermally robust, photo-reversible monomer to the polyester library only serves to further increase sustainability within the field.

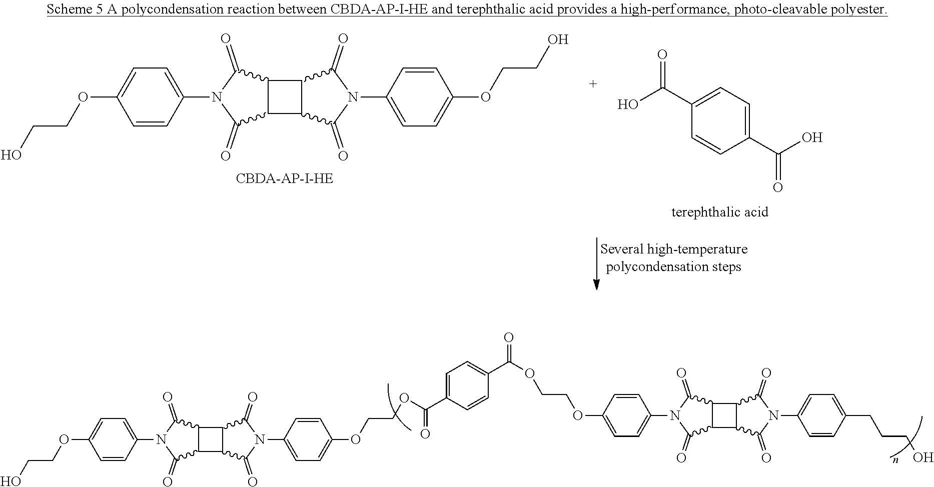

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed is:

1. A compound of Formula (I) or composition comprising the structure of Formula (I)

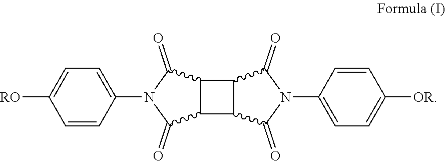

2. The compound or composition of claim 1, wherein R is selected from the group consisting of H, alkenyl, or hydroxyalkyl.

3. The compound or composition of claim 1, wherein the hydroxyalkyl is selected from the group consisting of C₁-C₁₂ hydroxyalkyl, and the alkenyl is selected from the group of C₁-C₁₂-alkenyl.

4. The compound or composition of claim 1, wherein the composition is selected form the group consisting of:

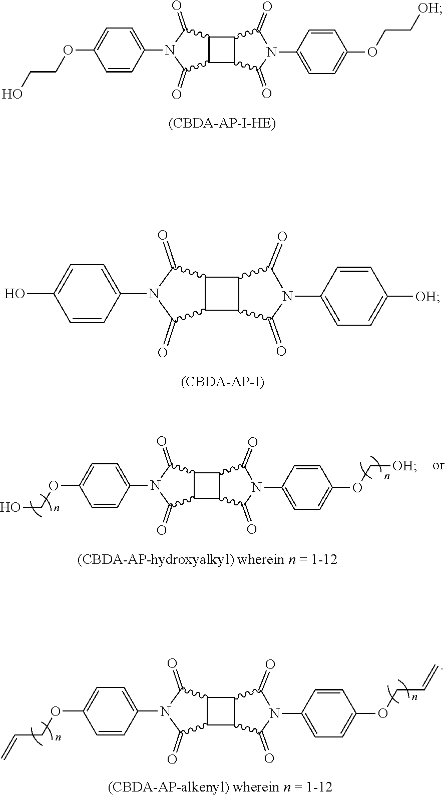

5. A polymer comprising the structure of Formula (I)

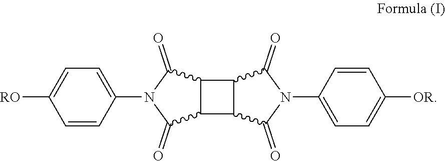

6. The polymer of claim 5, wherein R is selected from the group consisting of H, alkenyl, or hydroxyalkyl.

7. The polymer of claim 5, wherein the polymer comprises a repeating unit of 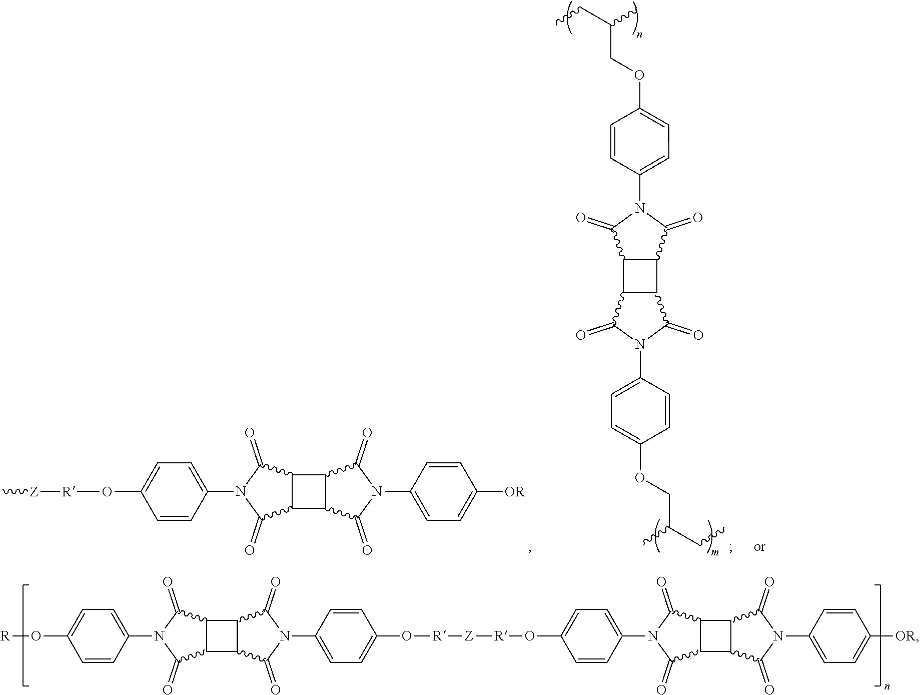
wherein, R is selected from the group consisting of H or hydroxyalkyl;
R′ is absent when R is H, or R′ is-O-alkyl when R is hydroxyalkyl; and
Z is a polyurethane, polyester, polysulfone, polyketone, or a monomer thereof.

8. The polymer of claim 5, wherein the polymer comprises about 1 mol. % to about 99 mol. % of the structure of Formula (I).

9. The polymer of claim 5, wherein the polymer further comprises at least one co-monomer.

10. The polymer of claim 5, wherein the co-monomer is selected from the group consisting of:
an isocyanate terminated monomer or polymer;
a di-carboxylic acid; and 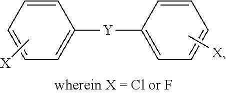
and
Y═SO₂ or C═O.

11. The polymer of claim 10, wherein the co-monomer is 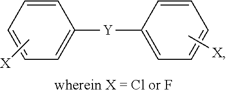
and
Y═SO₂ or C═O; and the polymer further comprises a diphenol co-monomer.

12. The polymer of claim 10, wherein the isocyanate terminated monomer or polymer is selected from the group consisting of:

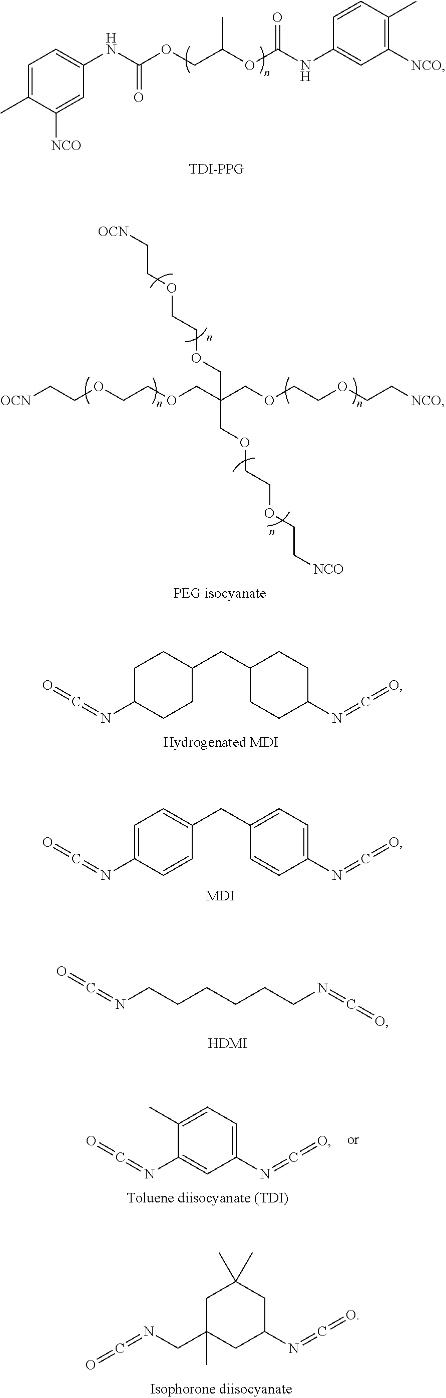

13. The polymer of claim 10, wherein the di-carboxylic acid is selected from the group consisting of 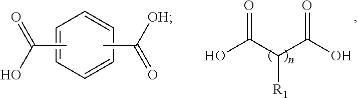
wherein R₁ is selected from the group consisting of H, alkyl, or aryl;

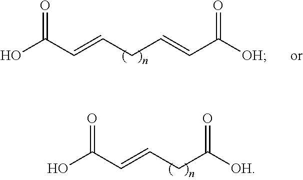

14. The polymer of claim 10, wherein the co-monomer is

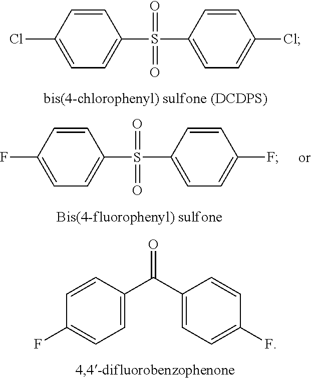 [SMILES: O=S(=O)(c1ccc(Cl)cc1)c1ccc(Cl)cc1 ; O=S(=O)(c1ccc(F)cc1)c1ccc(F)cc1 ; O=C(c1ccc(F)cc1)c1ccc(F)cc1]

15. The polymer of claim 10, wherein the diphenol is: 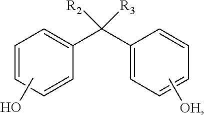
wherein R₂ and R₃ are independently selected from the group consisting of H and alkyl.

16. The polymer of claim 15, wherein the diphenol is

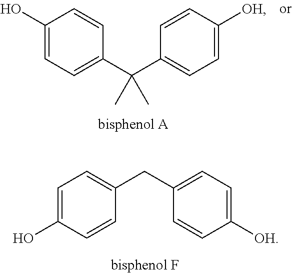 [SMILES: CC(C)(c1ccc(O)cc1)c1ccc(O)cc1 ; Oc1ccc(Cc2ccc(O)cc2)cc1]

17. The polymer of claim 5, wherein the polymer is a copolymer of the formula: 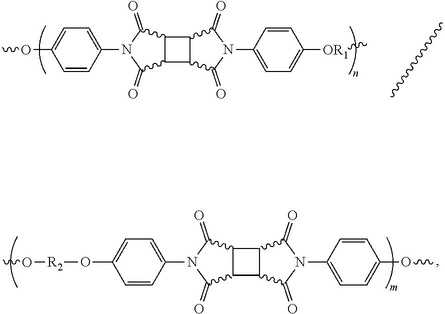
wherein R¹ and R₂ is any combination of a polyurethane, polysulfone, polyketone, polyester, or a monomer thereof.

18. A method of temporarily bonding a support substrate to a semiconductor wafer for processing, the method comprising:
obtaining a device wafer and a support substrate;
applying the photocleavable adhesive polymer of claim 5 to the surface of the support substrate, thereby forming an adhesive layer;
adhering the device wafer to the support substrate by placing it on top of the adhesive layer;
processing the device wafer, and
irradiating the adhesive layer with 254 nm light, thereby debonding the support substrate from the device wafer.

19. The method of claim 18, wherein the support substate comprises quartz or glass.

20. The method of claim 18, wherein the polymer is applied via spincoating.
